(19) United States
(12) Patent Application Publication
(10) Pub. No.: US 2008/0241084 A1
(43) Pub. Date: Oct. 2, 2008

(54) Compositions and methods for inhibiting melanogenesis

(75) Inventor: Mukhtar Siddiqui, San Ramon, CA (US)

Correspondence Address: KLARQUIST SPARKMAN, LLP, 121 SW SALMON STREET, SUITE 1600, PORTLAND, OR, 97204, US

(73) Assignee: Shaklee Corporation

(21) Appl. No.: 12/079,838

(22) Filed: Mar. 28, 2008

Related U.S. Application Data
(60) Provisional application No. 60/920,927, filed on Mar. 29, 2007.

(51) Int. Cl. A61K 8/97; A61Q 19/02

(52) U.S. Cl. 424/62

(57) ABSTRACT

A topical composition is disclosed that is effective in lightening the color of skin and/or inhibiting melanogenesis. The topical composition comprises Arctostaphylos uva ursi extract, Scutellaria extract, glycyrrhizic acid, Glycine soja extract, and Fagus sylvatica extract. The topical composition can inhibit melanogenesis in skin tissue without the concomitant adverse impact on cellular viability expected from these ingredients. In some cases, the topical composition further comprises a dermatological carrier such as, for example, a lotion or a cream, for administration to a subject's skin. Methods of making the topical composition are also disclosed along with methods of using the topical composition for skin lightening and inhibition of melanogenesis.

### CROSS REFERENCE TO RELATED APPLICATION

The present application claims the benefit of the earlier filing date of U.S. Provisional Application No. 60/920,927, filed Mar. 29, 2007, which is incorporated herein by reference.

### FIELD

The compositions and methods described herein related to dermatologic compositions useful for inhibiting melanogenesis and lightening the color of skin tissue.

### BACKGROUND

The color of human skin is differentiated by the amount of a natural pigment, melanin, and its distribution in the epidermal layers of skin. Melanin protects the body from the damaging effects of ultraviolet radiation by blocking and reflecting sunlight and ultraviolet radiation that can mediate harmful conditions. However, in some cases, it is desirable to reduce the amount of melanin in the skin, for example, to treat hyperpigmentary disorders. Hyperpigmentary disorders can cause embarrassment in those afflicted. Such disorders include, inter alia, freckles, solar lentigines (liver spots), and visible darkening that can occur in grafted skin.

Additionally, a reduction in the amount of melanin in the skin may be desired to lighten the skin for cosmetic reasons.

Melanin is produced within a population of cells called melanocytes located in the lower part of the epidermis. Within the melanocytes, an enzyme called tyrosinase mediates a process by which the amino acid tyrosine is converted to melanin. The melanin is deposited in melanosomes, which are vesicles found within the melanocytes. The melanosomes are extruded from the melanocytes and carried to the surface of the skin by keratinocytes, which internalize the melanin containing melanosomes. The darkness of the color observed in the skin is a function of the amount of melanin synthesized by melanocytes and transferred to the surface of the skin by keratinocytes. This process is known as melanogenesis.

Various means have been disclosed to reduce the amount of melanin present in hyperpigmented areas of the skin or for otherwise lightening the color of the skin. Many methods focus on inhibiting the tyrosinase mediated conversion of tyrosine to melanin. For example, U.S. Pat. No. 6,123,959 to Jones et al. discloses arbutin (a compound present in the leaves of the bearberry plant (Arctostaphylos uva ursi), licorice extract (from licorice root), ascorbic acid, and kojic acid as skin lightening agents that inhibit the tyrosinase mediated production of melanin. U.S. Pat. No. 5,980,904 to Leverett et al. discloses that hydroquinone, glutathione, cysteine, and mulberry extract (which also includes arbutin) provide such inhibition. U.S. Pat. No. 5,773,014 to Perrier et al. discloses a combination of mulberry, saxifrage, and grape and Scutellaria root as useful to inhibit tyrosinase activity. U.S. Pat. No. 6,348,204 to Touzan discloses a composition for skin whitening comprising at least one extract of mulberry, at least one extract of Scutellaria baicalensis, and at least one salicylic acid derivative. However, these compositions are expected to have adverse side effects, such as cytotoxicity or skin irritation since hydroquinone and arbutin (a derivative of hydroquinone) are cytotoxic and kojic acid has been reported to cause skin irritation. Kojic acid is also unstable and may lose efficacy upon exposure to air and sunlight.

Other methods of inhibiting melanogenesis involve inhibiting the transfer of melanin from melanocyte to keratinocyte. For example, Greatens et al. disclose that niacinamide can inhibit melanosome transfer and induce skin lightening. Experimental Dermatology, 14(7), 498-508 (July 2005). A further method involves inhibiting the action of a hormone that activates the melanin production of the melanocytes, as described in U.S. Pat. No. 5,126,327 to Takeuchi, et al.

Paine et al. disclose that both soymilk and soybean paste reduce melanin deposition within swine epidermis. Paine C. et al., An alternative approach to depigmentation by soybean extracts via inhibition of the PAR-2 pathway, J. Invest. Dermatol., 116:587-595 (2001). In some cases, skin lightening is accomplished without inhibiting melanogenesis, but rather by bleaching the area of skin desired to be lightened. Of course, bleaching preparations often irritate the skin.

### SUMMARY

In a surprising discovery, a topical composition comprising a formulation combining known inhibitors of the tyrosinase mediated production of melanin, Arctostaphylos uva ursi extract and Scutellaria extract, with glycyrrhizic acid, Glycine soja extract, and Fagus sylvatica extract inhibited melanogenesis in skin tissue without the concomitant adverse impact on cellular viability expected from the use of these ingredients. Thus, the present disclosure provides a topical composition comprising a melanogenesis inhibiting formulation comprising Arctostaphylos uva ursi extract, Scutellaria extract, glycyrrhizic acid, Glycine soja extract, and Fagus sylvatica extract. In some cases, this disclosed melanogenesis inhibiting formulation is further combined in the topical composition with a dermatological carrier such as, for example, a lotion or a cream, for administration to a subject's skin. The topical composition in some embodiments further comprises other skin treatment and/or protective agents. Also provided are methods of making such compositions. Further, as compositions comprising the disclosed melanogenesis inhibiting formulation are useful for inhibiting melanogenesis and lightening the color of the skin, the present disclosure also provides methods of using the disclosed topical composition for inhibiting melanogenesis and lightening the color of skin, as well as treating or preventing any condition for which melanogenesis inhibition and or skin lightening is indicated.

The foregoing and other objects, features, and advantages of the disclosed invention will become more apparent from the following detailed description, which proceeds with reference to the accompanying FIGURE.

### BRIEF DESCRIPTION OF THE FIGURE

FIG. 1 is a graph showing the relative efficacy of the various test articles tested in Example 1 in inhibiting melanogensis in skin tissue, relative to a negative control. (GS is an aqueous solution comprising 1% Glycine soja extract by weight; SB is an aqueous solution comprising 1% Scutellaria baicalensis extract by weight; A is an aqueous solution comprising 1% Arctostaphylos uva ursi extract by weight; FS is an aqueous solution comprising 1% Fagus sylvatica extract by weight; DG is an aqueous solution comprising 1% dipotassium glycyrrhizate by weight; Mix is an embodiment of the disclosed topical composition comprising, by weight, 0.2% of each of Arctostaphylos uva ursi extract, Scutellaria baicalensis extract, dipotassium glycyrrhizinate, Glycine soja extract, and Fagus sylvatica extract.

### DETAILED DESCRIPTION

The term “subject” means an animal, such as a mammal, for example a human.

The term “extract” refers to preparation comprising soluble plant components obtained by means of a solvent (including a mixture of solvents) from a plant (or portions thereof), optionally under pressure, ultrasonic, or at a particular temperature or range of temperatures. Suitable solvents include water, organic solvents, such as alcohol and/or butylene glycol or propylene glycol, and supercricital fluids. An extract extracted with a particular solvent is referred to as a “[solvent] extract,” for example, a water extract, or an alcohol extract. Those of ordinary skill in the art are knowledgeable concerning many methods of producing a plant extract. Suitable techniques are described in U.S. Pat. Nos. 5,891,440, 5,874,084, and 5,908,628. A typical method of making an extract includes grinding up plant material (for example the flowers or root of a plant) with a grinding device, such as a mortar and pestle, placing the plant material into a container, adding a solvent such as water or alcohol to the container, allowing the plant material and solvent to sit for a period of time, such as overnight, and filtering out the plant material from the solvent to leave an extract. In some cases the solvent is evaporated to leave a concentrated extract, for example by heating the plant/solvent mixture to a particular temperature, such as about 35-45° C., optionally under vacuum, such as about negative 500 to about negative 1000 bar. In other cases, the solvent is not evaporated, and the extract comprises the soluble plant components and the solvent. In some cases the extract additionally comprises other ingredients such as stabilizers, preservatives, and/or antimicrobial agents which are often used reduce loss of efficacy of the extract as well as to prevent the growth of bacteria and mold while it is being stored prior to its use and/or combination with other ingredients into a composition. Such stabilizers, preservatives, and/or antimicrobial agents include, for example, water, the parabens (such as the methyl, ethyl, propyl, isopropyl, butyl and isobutyl esters), imidazolidinyl urea, diazolidinyl urea, quaternium-15, phenylethyl alcohol, benzyl alcohol, phenoxyethanol, chlorphenesin, chlorhexidine digluconate as well as combinations of these agents. In specific cases an extract comprises the anti-microbial stabilizer Phenonip® (a stabilizing composition comprising paraben esters in phenoxyethanol from Clariant used to stabilize cosmetic formulations).

Lightening of the color of skin refers to any visible change in the color or tone of skin tissue from a darker state to a lighter state.

The disclosed melanogenesis inhibiting formulation comprises Arctostaphylos uva ursi extract, Scutellaria extract, glycyrrhizic acid, Glycine soja extract, and Fagus sylvatica extract.

An Arctostaphylos uva ursi extract comprises an extract from the plant material of the Arctostaphylos uva ursi plant, commonly known as the bearberry plant. Extracts of this plant inhibit the tyrosinase mediated production of melanin. The active ingredient in Arctostaphylos uva ursi believed to cause inhibition of the tyrosinase mediated production of melanin is the glycoside arbutin. Thus, in certain embodiments an Arctostaphylos uva ursi extract comprises an extract that contains arbutin. Arbutin is found in the leaves of the uva ursi plant. Thus, in certain embodiments, an Arctostaphylos uva ursi extract comprises an extract from the leaves of the uva ursi plant. An Arctostaphylos uva ursi extract in certain embodiments comprises an extract extracted with water, an extract extracted with an organic solvent, such as, for example alcohol, butylene glycol, or a combination thereof. In a specific embodiment the Arctostaphylos uva ursi extract is a water extract from the leaf of the plant. An Arctostaphylos uva ursi extract in some cases further comprises stabilizers, preservatives, and/or antimicrobial agents. In a specific embodiment the Arctostaphylos uva ursi extract comprises 20% of an extract the Arctostaphylos uva ursi plant, 39.5% water, 39.5% butylene glycol, and 1% Phenonip®. Arctostaphylos uva ursi extract is commercially available from numerous commercial sources, such as for example, Active Organics or Lewisville, Tex. Active Organics sells an Arctostaphylos uva ursi extract under the name Actiphyte® of Bearberry BG50P.

A Scutellaria extract is an extract from the plant material of a member of the Scutellaria genera. Useful plants in the Scutellaria genera include the known medicinal species Scutellaria baicalensis, Scutellaria galericulata, and Scutellaria lateriflora. In particular embodiments of the disclosed topical composition the Scutellaria extract is selected from the group consisting of a Scutellaria baicalensis extract, a Scutellaria galericulata extract, or a Scutellaria lateriflora extract or combinations thereof, such as for example, a Scutellaria baicalensis extract. Extracts of this plant inhibit the tyrosinase mediated production of melanin. In certain embodiments, a Scutellaria extract is an extract from the root of the Scutellaria plant. A Scutellaria extract in certain embodiments is an extract extracted with water, an extract extracted with an organic solvent, such as, for example alcohol, or a combination thereof. In a specific embodiment, the Scutellaria extract is a Scutellaria baicalensis extract that is a water extract from the root of the plant. In certain cases the Scutellaria baicalensis extract comprises 20% of an extract the Scutellaria baicalensis plant, 39.5% water, 39.5% butylene glycol, and 1% Phenonip®. Scutellaria baicalensis extract is available from numerous commercial sources, such as, for example, Active Organics or Lewisville, Tex. Active Organics sells an Scutellaria baicalensis extract under the name Actiphyte® of Skull Cap BG50P.

Glycyrrhizic acid is a compound found in the root of the licorice plant (Glycyrrhiza glabra). As used herein, glycyrrhizic acid includes that acid as well as derivatives thereof and/or salts thereof such as dipotassium glycyrrhizinate and monoammonium glycyrrhizinate. It is commercially available in powdered form from Mafco Worldwide Corporation. In a specific embodiment the glycyrrhizic acid is dipotassium glycyrrhizate, in the form of a powder comprising about 96% or more of glycyrrhizic acid, e.g., 96% to about 100% glycyrrhizic acid.

Glycine soja extract is an extract from the plant material of the soybean plant. In certain embodiments, a Glycine soja extract is an extract from the seeds of the Glycine soja plant, commonly called soybeans. A Glycine soja extract in certain cases is an extract extracted with water, an extract extracted with an organic solvent, such as, for example alcohol, or a combination thereof. In a specific embodiment the Glycine soja extract is a water extract from the seeds of the Glycine soja plant, commonly known as soy milk. In a further specific embodiment the Glycine soja extract comprises 20% soy milk, 39.5% water, 39.5% butylene glycol, and 1% Phenonip®. Glycine soja extract is available from numerous commercial sources, such as, for example, Active Organics of Lewisville, Tex. Active Organics sells a Glycine soja extract under the name Actiphyte® of Soybean BG50P.

Fagus sylvatica extract is an extract from the plant material of the Fagus sylvatica plant, commonly known as a beech tree. In certain embodiments, a Fagus sylvatica extract is an extract from the buds of the Fagus sylvatica plant. A Fagus sylvatica extract in certain embodiments is an extract extracted with water, an extract extracted with an organic solvent, such as, for example, alcohol, or a combination thereof. In a specific embodiment, the Fagus sylvatica extract is a water extract from the buds of the Fagus sylvatica plant. Fagus sylvatica extract is available from numerous commercial sources, such as for example, Gattefosse of France. Gattefosse sells a Fagus sylvatica extract under the name Gatuline RC, which comprises an extract from the Fagus sylvatica plant and water.

The disclosed melanogenesis inhibiting formulation comprising Arctostaphylos uva ursi extract, Scutellaria extract, glycyrrhizic acid, Glycine soja extract, and Fagus sylvatica extract comprises a sufficient amount of each ingredient to inhibit melanogenesis in a subject and/or lighten the subject's skin when the formulation is applied to the subject's skin. In certain embodiments this formulation further comprises a combination of each ingredient in relative amounts that cause the formulation to inhibit melanogenesis in a subject and/or lighten the subject's skin without decreasing the cellular viability of the subject's skin. For example, in certain embodiments the formulation comprises about 1%-75% Arctostaphylos uva ursi extract, about 1%-75% Scutellaria extract (e.g., a Scutellaria baicalensis extract), about 1%-75% glycyrrhizic acid, about 1%-75% Glycine soja extract, and about 1%-75% Fagus sylvatica extract by weight in relative amounts such that the total percentage of the five ingredients in the disclosed melanogenesis inhibiting formulation is 100%. In some cases the formulation comprises about 5%-35% Arctostaphylos uva ursi extract, about 5%-35% Scutellaria extract (e.g., a Scutellaria baicalensis extract), about 5%-35% glycyrrhizic acid, about 5%-35% Glycine soja extract, and about 5%-35% Fagus sylvatica extract. In more particular embodiments, the formulation comprises about 15%-25% Arctostaphylos uva ursi extract, about 15%-25% Scutellaria extract (e.g., a Scutellaria baicalensis extract), about 15%-25% glycyrrhizic acid, about 15%-25% Glycine soja extract, and about 15%-25% Fagus sylvatica extract. In a still further embodiment the formulation comprises equivalent percentages by weight of the five ingredients, such that the formulation comprises about 20% Arctostaphylos uva ursi extract, about 20% Scutellaria extract (e.g., a Scutellaria baicalensis extract), about 20% glycyrrhizic acid, about 20% Glycine soja extract, and about 20% Fagus sylvatica extract. In other specific embodiments the disclosed melanogenesis inhibiting formulation comprises about 12% Arctostaphylos uva ursi extract, about 24% Scutellaria baicalensis extract, about 24% glycyrrhizic acid, about 12% Glycine soja extract, and about 49% Fagus sylvatica extract. In certain cases this embodiment is included in a cleansing foam or cleansing gel composition, such as is disclosed in Examples 6-7. In still other embodiments the disclosed melanogenesis inhibiting formulation comprises about 15% Arctostaphylos uva ursi extract, about 3% Scutellaria extract (e.g., a Scutellaria baicalensis extract), about 3% glycyrrhizic acid, about 15% Glycine soja extract, and about 63% Fagus sylvatica extract. In some cases this embodiment is included in a cream, day lotion, or essence composition, such as is disclosed in Examples 2-4. In yet another embodiment the disclosed melanogenesis inhibiting formulation comprises about 14% Arctostaphylos uva ursi extract, about 3% Scutellaria extract (e.g., a Scutellaria baicalensis extract), about 14% glycyrrhizic acid, about 14% Glycine soja extract, and about 56% Fagus sylvatica extract. In some cases this embodiment is included in a toner/lotion composition such as is disclosed in Example 5. In still further embodiments, the disclosed melanogenesis inhibiting formulation comprises about 45% Arctostaphylos uva ursi extract, about 1% Scutellaria extract (e.g., a Scutellaria galericulata extract), about 5% glycyrrhizic acid, about 5% Glycine soja extract, and about 45% Fagus sylvatica extract. In some cases this embodiment is included in a spot treatment composition, such as is disclosed in Example 10(a).

In some cases the extracts comprising the formulation are preparations of which only a portion of the extract is the plant extract. For example, the various extracts in some cases comprise about 20% of extract from the relevant plant with the remainder of the extract comprising stabilizers, preservatives, and/or antimicrobial agents, as described above. Thus, in certain cases, the melanogenesis inhibiting formulation comprises 20% Arctostaphylos uva ursi extract, which comprises 20% of an extract from the Arctostaphylos uva ursi plant, 39.5% water, 39.5% butylene glycol, and 1% Phenonip®; 20% Scutellaria baicalensis extract, which comprises 20% of an extract from the Scutellaria baicalensis plant, 39.5% water, 39.5% butylene glycol, and 1% Phenonip®; 20% Glycine soja extract, which comprises 20% soy milk, 39.5% water, 39.5% butylene glycol, and 1% Phenonip®; and 20% of an extract from the Fagus sylvatica plant. In other embodiments the melanogenesis inhibiting formulation comprises Arctostaphylos uva ursi extract, Scutellaria extract (e.g., a Scutellaria baicalensis extract), Glycine soja extract, and Fagus sylvatica extract wherein each extract comprises anywhere from about 1% to about 100% of an extract from the relevant plant.

In certain embodiments the disclosed melanogenesis inhibiting formulation is produced by combining Arctostaphylos uva ursi extract, Scutellaria extract (e.g., a Scutellaria baicalensis extract), glycyrrhizic acid, Glycine soja extract, and Fagus sylvatica extract and mixing these ingredients together. The ingredients are combined in any suitable container, such as a mixing vessel, in any order, and in any of the amounts described herein.

In some embodiments, the disclosed topical composition comprising the disclosed melanogenesis inhibiting formulation further comprises a dermatologically acceptable carrier. Dermatologically acceptable carriers are those substances, which, as is known, can be used in the field of dermatology, pharmacology, cosmetics, and related fields, and other substances whose properties do not impede physiological use when applied to the skin.

In still further cases the disclosed topical composition further comprises any other ingredients that are generally considered safe for topical application to skin, such as other skin lightening ingredients, antioxidants, anti-inflammatory agents and other ingredients.

In embodiments where the disclosed topical composition further comprises a dermatologically acceptable carrier or other ingredients, the disclosed melanogenesis inhibiting formulation comprises from about 0.1% to about 99.9% by weight of the resulting composition, depending on the degree of efficacy in melanogenesis inhibition and/or skin lightening desired. Thus, in certain of such embodiments, the disclosed melanogenesis inhibiting formulation comprises, by weight of the resulting composition, about 0.1%-0.5%, for example about 0.3%-0.5%, such as about 0.36% (as in Example 5) or 0.41% (as in Examples 6-7), about 0.5%-5%, such as, for example, about 1% (as in Example 1), about 2.2% (as in Example 10(a)), or about 3.2% (as in Examples 2-4), about 5%-10%, about 10%-15%, or about 15%-20%. In certain embodiments the disclosed melanogenesis inhibiting formulation comprises, by weight about 0.35%-3.2% of the topical composition.

In such compositions where the disclosed topical composition further comprises a dermatologically acceptable carrier, the ingredients of the disclosed melanogenesis inhibiting formulation comprise percentages by weight of the topical composition, such as, for example, the Arctostaphylos uva ursi extract comprises from about 0.05% (as in Examples 5-7) to about 0.5% (as in Examples 2-4) to about 1% (as in Example 10(a)), the Scutellaria extract (e.g., a Scutellaria baicalensis extract or Scutellaria baicalensis extract) comprises from about 0.01% (as in Examples 5-7) to about 0.2% (as in Examples 1 and 10(a)), the glycyrrhizic acid comprises from about 0.05% (as in Examples 5-7) to about 0.1% (as in Example 10(a)) to about 0.2% (as in Example 1), the Glycine soja extract comprises from about 0.05% (as in Examples 5-7) to about 0.1% (as in Example 10(a)) to about 0.5% (as in Examples 2-4), and Fagus sylvatica extract comprises from about 0.2% (as in Examples 1 and 5-7) to about 1% (as in Example 10(a)) to about 2% (as in Examples 2-4).

Suitable dermatologically acceptable carriers include lubricants, wetting agents, emulsifying and suspending agents, preservatives, antiirritants, emulsion stabilizers, film formers, gel formers, odor masking agents, resins, humectants, hydrocolloids, solvents, solubilizers, neutralizing agents, permeation accelerators, pigments, quaternary ammonium compounds, detergents and cleansing agents, surfactants, sequestering agents, refatting and superfatting agents, ointment, cream or oil base materials, silicone derivatives, stabilizers, sterilizing agents, propellants, drying agents, sunscreen agents, opacifiers, thickeners, waxes, skin conditioning agents, emollients, and/or white oils, and combinations of these ingredients.

Possible surfactants include polyoxyethylene sorbitan esters of fatty organic acids (such as laureate, palmitate, stearate, oleate and myristate) containing various molar concentrations of ethylene oxide (commonly listed as polysorbate 20, 21, 40, 60, 61, 65, 80, 81 and 85) as well as combinations of these ingredients. Possible surfactants further include the salts of fatty acids (such as sodium, potassium, amine or amino acid salts of stearic, myristic, oleic, lauric or palmitic acid), non-ionic surfactants such as the polysorbates listed above, sorbitan esters of fatty acids (such as stearates, myristates, oleates, laureates, and palmitates), glyceryl esters of fatty acids (stearate, myristate, oleate, laureate and palmitate), polyoxyethylene esters of lanolin acids, alcohols and other wool wax components, polyoxyethylene ethers of fatty alcohols (such as lauryl, cetyl, oleyl and stearyl), polyethylene glycol esters of fatty acids (such as laureate, stearate, myristate, oleate, and palmitate), homo- and mixed block polymers of polyoxyethylene and polyoxypropylene, polyoxypropylene esters of fatty acids, polyoxypropylene ethers of fatty alcohols, sugar esters of fatty acids (such as the fatty acid esters of glucose and sucrose).

Possible humectants include sugars (such as sorbitol, glucose, etc.), glycerin (and its polymers), glycols (such as propylene glycol, butylene glycol, and polyethylene glycols of various molecular weights), hyaluronic acid (and its salts), pyrrolidone carboxylic acid (and its salts) as well as combinations of these ingredients.

Possible preservatives include the parabens (such as the methyl, ethyl, propyl, isopropyl, butyl and isobutyl esters), imidazolidinyl urea, diazolidinyl urea, quaternium-15, phenylethyl alcohol, benzyl alcohol, phenoxyethanol, chlorphenesin, chlorhexidine digluconate as well as combinations of these ingredients.

Possible sequestering agents include the various salts of ethylenediamine tetraacetic acid (sodium, potassium, amine and amino acid salts).

Possible emollients include esters of fatty acids and fatty alcohols (such as octyl palmitate, octyl stearate, cetearyl stearate, etc.), silicone compounds (such as dimethicone, cyclomethicone, phenyltrimethicone, etc.), esters of organic acids and organic alcohols (C12-15 alkyl benzoate, octyl dodecanol, cetyl lactate, tridecyl trimellitate, octyldodecyl neopentanoate, etc.), fatty alcohols (cetyl alcohol, stearyl alcohol, etc.), castor oils, fractions of castor oils and their hydrogenated derivatives as well as combinations of these types of ingredients.

Possible thickeners include acrylic acid polymers and their cross polymer derivatives, polyvinylpyrrolidone polymers, natural polymers (such as locus bean gum, xanthan gum, alginic acid and its salts, dextran, etc.), clays (hectorite, montmorillonite, etc.) as well as combinations of these ingredients.

Possible emulsifiers include cetyl dimethicone copolyols and other various other dimethicone copolyols in addition to combinations of these ingredients.

Possible sunscreens include any approved sunscreen agents such as dioxybenzone, homomethyl salicylate, menthyl anthranilate, octocrylene, octyl methoxycinnamate, octyl paraminobenzoate, octyl salicylate, oxybenzone, and trolamine salicylate, as well as combinations of these ingredients.

Possible detergents and cleansing agents include the salts of cocyl isethionate, isostearoyl lactylate salts (such as the sodium and potassium salts), tallow and tallow salts (such as sodium, potassium and ammonium salts), salts of lauryl and laureth sulfates (such as sodium, potassium and ammonium salts), betaines and sultaines (such as cocamidopropyl betaine or sultaine) and salts of fatty acids (such as sodium or potassium laureate, myristate, palmitate, stearate, oleate, behenate, linoleate and ricinoleate) as well as combinations of these ingredients.

Possible skin conditioning agents include the humectants listed above, salts of isostearoyl lactylate (such as sodium or potassium), quaternium compounds (such as stearamidopropyl dimethylamine) and oat by-products (such as oat flour) as well as combinations of these ingredients.

In certain embodiments, the dermatologically acceptable carrier comprises from about 0.1% to about 99.9% by weight of the disclosed topical composition. In certain embodiments, the carrier comprises between about 50% and about 99% of the composition by weight. In further embodiments, the carrier comprises about 60% to about 70% of the composition, about 70% to about 80% of the composition, about 80% to about 90% of the composition, about 90% to about 99% of the composition, or about 99% to about 99.9% of the composition (all % by weight of the total weight of the composition).

The ingredients comprising the topical composition are combined by any suitable method. For example, in some cases, the ingredients of which the disclosed melanogenesis inhibiting formulation is comprised are first added to each other, following which the disclosed melanogenesis inhibiting formulation is simply added to one or more of the ingredients comprising the dermatologically acceptable carrier. In other cases, the ingredients of which the disclosed melanogenesis inhibiting formulation is comprised are added separately to one or more of the ingredients comprising the dermatologically acceptable carrier. In certain cases the melanogenesis inhibiting formulation and one or more of the ingredients comprising the dermatologically acceptable carrier are mixed together, for example, by stirring and/or agitation.

The disclosed topical composition in some cases is provided in a container from which it may be administered. Such a container includes, for example, a squeeze tube, a mister, a bottle, or a foil package.

The disclosed topical composition is useful in methods of preventing and/or treating conditions involving excess melanin production including solar and simple lentigines (including age/liver spots), melasma/chloasma and postinflammatory hyper-pigmentation. The disclosed topical composition is believed to reduce skin melanin levels by, among other means, inhibiting the production of melanin, whether it is produced constitutively or in response to UV irradiation (such as sun exposure). Thus, the disclosed topical composition is also useful in methods of reducing skin melanin content in non-pathological states so as to induce a lighter skin tone, as desired by the user, or to prevent melanin accumulation in skin that has been exposed to UV irradiation. The disclosed topical composition is also useful for lightening skin color without regard to its mechanism of action.

As discussed herein, the disclosed topical composition in certain embodiments lightens skin and inhibits melanin production without a reduction in skin cell viability and, in some cases, with an increase in skin cell viability. Thus, the disclosed topical composition is also useful for lightening skin and/or inhibiting melanin production in a subject without mediation of a decrease in cell viability and/or to increase cell viability.

In methods of using the disclosed topical composition, the composition is administered topically to the skin of a subject in an effective amount to inhibit melanogenesis and/or lighten the color of the skin (and, in certain embodiments, in an amount that does not decrease cell viability of the treated skin cells, or which increases the viability of such cells). Topical administration provides the topical composition directly to the skin. Topical administration preferably comprises applying the disclosed topical composition in the form of a composition comprising a dermatologically acceptable carrier as described above as well as in the Examples herein. It is generally preferred that the carrier comprise a material which will form a film or layer on the skin to which it is applied. This localizes the application and provides some resistance to perspiration and/or aids in percutaneous delivery and penetration of active ingredients into the skin. Many such compositions are known in the art, and can take the form of creams, gels, ointments, hydrogels, pastes or plasters, and liquid dosage forms, such as solutions, emulsions, in particular oil-in-water emulsions, suspensions, for example lotions etc., or even solid sticks.

An effective amount of the disclosed topical composition is such an amount as is capable of inhibiting melanogenesis and/or lightening the color of the skin (and, in certain cases, which also does not decrease cell viability in the treated skin, or increases the viability of such cells). The exact amount required will vary from case to case, depending on variables such as the amount of skin to be treated, the amount of lightening desired, the amount of the melanogenesis inhibiting formulation relative to the amount of the dermatologically acceptable carrier, if such a carrier is employed, the amount of extract from the relevant plant in the extracts that comprise the melanogenesis inhibiting formulation, the number of and schedule of the doses of the disclosed topical composition to be administered, the degree of skin lightening desired, the desired decrease in melanin produced, and other variables relevant to an individual subject being treated. Thus, it is not possible to specify an exact “effective amount.” An appropriate effective amount may be determined by one of ordinary skill in the art using only routine experimentation in combination with the disclosure herein. However, in some cases, an effective amount of the topical composition is a dose of about 1 mg/cm² to about 40 mg/cm² of skin to which the topical composition is applied. Dose refers to a quantity of the topical composition applied at one time. In certain embodiments an effective amount is a dose of about 2 mg/cm² of skin.

Further, one of ordinary skill in the art will appreciate that the endpoint for any of the methods of inhibiting melanogenesis and/or lightening the skin disclosed herein will vary according to the disease, condition, or disorder being treated, the outcome desired by the subject being treated and/or individual administering the treatment, and other factors. Thus, the methods of using the topical composition disclosed herein contemplate the application of one or multiple does of the topical composition. In some cases a first dose is applied to a subject's skin and one or more subsequent doses are applied at a later time or times, such as later in the same day, or on subsequent days.

Where the disclosed topical composition is being used to lighten skin color such as, for example, to reverse hyperpigmentation caused by, for example, inflammation or diseases such as melasma, any one of a number of endpoints can be chosen. For example, endpoints can be defined subjectively such as, for example, when the subject or individual administering the composition, such as a treating physician, is “satisfied” with the results of administration of the disclosed topical composition. Alternatively, endpoints can be defined objectively. For example, the subject's skin in the treated area can be compared to a color chart or an already lighter reference area of his or her skin, which, in some cases, is the subject's normal skin color. Treatment is continued, with subsequent doses applied, until the color of the skin in the treated area is the same or similar in appearance to a color on the chart and/or the color of the reference area of the subject's skin. A subject's normal skin color can be determined by observing the subject's skin in areas that are not hyperpigmented. Alternatively, the reflectance of the treated skin can be measured, and treatment can be terminated when the treated skin attains a specified reflectance or, e.g., a specified decrease in reflectance or decrease in skin color as measured by e.g., the skin tan value (i.e., ITA°, which is inversely correlated to skin color). For example, the treatment could be discontinued when the ITA° of a hyperpigmented or colored area of a subject's skin has increased by about 10%-100%, for example, about 20% or more, such as 20-30%, or 40-50%, or 60-70%, or more. In certain cases, the ITA° of a hyperpigmented area of a subject's skin has been increased by about 29% during a four week regimen of administering the disclosed topical composition, by about 41% during an eight week regimen of administering the disclosed topical composition, and by about 69% during a twelve week regimen of administering the disclosed topical composition (see Example 9). Alternatively, the melanin content of the treated skin can be measured. Treatment can be terminated when the melanin content of the treated skin reaches a specified value. Melanin content can be determined in any way known to the art, including by histological methods, with or without enhancement by stains for melanin.

### EXAMPLE 1

This Example provides the results of a study of the melanogenesis inhibitory and skin lightening effects of the disclosed topical composition. The study compares the disclosed topical composition with a negative control, a positive control that is a known inhibitor of melanogenesis, as well as each of the ingredients that are combined in the disclosed melanogenesis inhibiting formulation, but on an individual basis. The results demonstrate the effectiveness of the disclosed topical composition in inhibiting melanogenesis and lightening the skin. The results further, and surprisingly, demonstrate that even though the individual ingredients in the disclosed topical composition reduce the cell viability of treated tissues, when the ingredients are combined, the resulting topical composition does not decrease cell viability and, indeed, increased cell viability (relative to the negative control). The study was performed by MatTek Corporation on behalf of Shaklee Corporation.

The articles tested were:

GS—an aqueous solution comprising 1% Glycine soja extract that comprised 20% soy milk, 39.5% water, 39.5% butylene glycol, and 1% Phenonip®;

SB—an aqueous solution comprising 1% Scutellaria extract that comprised 20% of an extract from an Scutellaria baicalensis plant, 39.5% water, 39.5% butylene glycol, and 1% Phenonip®;

A—an aqueous solution comprising 1% Arctostaphylos uva ursi extract that comprised 20% of an extract from an Arctostaphylos uva ursi plant, 39.5% water, 39.5% butylene glycol, and 1% Phenonip®;

FS—an aqueous solution comprising 1% Fagus sylvatica extract;

DG—an aqueous solution comprising 1% dipotassium glycyrrhizate;

Mix—an aqueous solution comprising 1% by weight of the disclosed topical composition, wherein the composition comprises 0.2% by weight of each of: 1) an Arctostaphylos uva ursi extract, which comprises 20% of an extract from an Arctostaphylos uva ursi plant, 39.5% water, 39.5% butylene glycol, and 1% Phenonip®; 2) a Scutellaria extract, which comprises 20% of an extract from a Scutellaria baicalensis plant, 39.5% water, 39.5% butylene glycol, and 1% Phenonip®; 3) dipotassium glycyrrhizate; 4) Glycine soja extract, which comprises 20% soy milk, 39.5% water, 39.5% butylene glycol, and 1% Phenonip®; and 5) an extract from a Fagus sylvatica plant.

Also tested were:

Positive control—1% by weight kojic acid (KA) dissolved in sterile ultra pure water (18 MOhm); and

Negative control—sterile ultrapure water (H₂O).

In this study, the disclosed topical composition and the other tested substances were introduced in vitro to epidermal equivalent systems. Then the effects of the substances on cell viability, skin color, melanin production, melanocyte morphology and dendricity and tissue morphology were determined.

### Cell Viability

The effect of the tested articles on cell viability was determined with an MTT assay. 10 μL and 25 μL portions of each of GS, SB, A, FS, DG and Mix were applied to separate duplicate samples of EpiDerm tissue (e.g., GS was applied to four tissue samples, with two samples receiving 10 μL and two samples receiving 25 μL of GS). 25 μL of the negative control was applied to each of three separate samples of EpiDerm tissue. EpiDerm tissue is an epidermal equivalent system from the MatTek Corporation of Ashland, Mass., consisting of normal, human-derived epidermal keratinocytes (NHEK) which have been cultured to form a multilayered, highly differentiated model of the human epidermis. After 48 hours of exposure to the test article or negative control each sample was rinsed with phosphate buffered saline (PBS) and the viability of the tissue (referenced to the negative control) was determined using the MTT tissue viability assay.

The MTT assay determines cell viability by measuring the amount of 3-(4,5-dimethylthiazol-2-yl)-2,5-diphenyltetrazolium bromide (MTT) dye taken up by the cell cultures. Viable cells take up this dye and convert it to insoluble formazin crystals that resides in the mitochondria of the cells until extracted with alcohol. The amount of MTT converted to extractable formazin crystals is directly proportional to the viability of the cell culture. MTT was measured spectrophotometrically and the optical density of each sample was determined. The mean optical density of the negative control (water in this case) is used as 100% cell viability. The percent cell viability resulting from each test article is determined by dividing the mean optical density of the samples exposed to each test article at each testing amount (10 or 25 μL) by the mean optical density of the samples exposed to the negative control and multiplying by 100.

The results of the cell viability test are provided in Table 1. As can be seen, each of the separate ingredients that are combined in the embodiment of the disclosed topical composition tested decreased cell viability at one or both of the amounts tested. Thus, one would expect that the disclosed topical composition would also decrease cell viability. However, surprisingly, the embodiment of the disclosed topical composition tested (Mix) did not have a negative effect on cell viability. Indeed, the Mix resulted in greater cell viability than the negative control.

TABLE 1

| Test | % Viability of tissue sample |
|---|---|
| Article | exposed to test article |
| GS(10 mL) | 96.12% |
| GS(25 mL) | 88.84% |
| SB(10 mL) | 101.27% |
| SB(25 mL) | 95.09% |
| A(10 mL) | 97.63% |
| A(25 mL) | 87.82% |
| FS(10 mL) | 97.47% |
| FS(25 mL) | 111.31% |
| DG(10 mL) | 94.62% |
| DG(25 mL) | 94.46% |
| MIX(10 m | 120.09% |
| MIX(25 m | 124.21% |

### Skin Color, Melanin Production Melanocyte Morphology and Dendricity and Tissue Morphology

The effects of the test articles on skin color, melanin production, melanocyte morphology and dendricity and tissue morphology were assessed using MelanoDerm tissue (MEL-300-B). MelanoDerm tissue is another epidermal equivalent system from the MatTek Corporation of Ashland, Mass. This system consists of normal, human-derived epidermal keratinocytes (NHEK) and melanocytes (NHM) which have been cultured to form a multilayered, highly differentiated model of the human epidermis. The NHM within co-cultures undergo spontaneous melanogenesis leading to tissue pigmentation. The MEL-300-B system contains melanocytes derived from a black donor.

On Day zero of the study, prior to exposure to any test articles, MelanoDerm tissue samples were placed into wells on plates containing maintenance medium that had been pre-warmed to about 37° C. (Long life maintenance medium, EPI-100-LLMM). The Melanoderm tissue samples were then incubated for about an hour in a humidified incubator maintained at a temperature of about 37° C. in an atmosphere of about 5% CO₂. Following incubation, the maintenance medium was removed by aspiration. A cell culture stand was then placed in each well on the plate containing a MelanoDerm tissue sample. About 5 mL of fresh maintenance medium pre-warmed to about 37° C. was then added to each well having a MelanoDerm tissue sample. Then a cell culture insert was placed on top of each cell culture stand. 25 μL portions of each of GS, SB, A, FS, DG, Mix, negative control, and positive control were applied by pipette to six separate MelanoDerm tissue samples for each test article being tested (e.g., GS was applied to six tissue samples in six different wells). 25 μL portions of each of the test articles and controls were also applied to the same MelanoDerm tissue samples on Days, 1, 3, 6, and 8 of the study for each sample remaining (some samples were used for testing). Throughout the study, the maintenance medium was changed every two days to maintain the MelanoDerm tissue samples.

On each of Days 3, 7, 10, and 14, one tissue sample for each test article and control was fixed with formalin, photographed to document macroscopic darkening, microscopically observed to examine melanocyte morphology and dendricity, then processed for histology. On Days 10 and 14, two tissue samples for each test article and control were frozen and later assayed for melanin content.

To assay the tissues for melanin content, the frozen tissues were thawed and placed in Dulbecco's phosphate buffered saline (D-PBS) to remove excess phenol red from the maintenance medium. The tissues were removed from the D-PBS and each tissue was blotted dry and placed in a separate 1.7 ml microfuge tube. To each microfuge tube 250 μl of a tissue and gel solubilizer was added (Solvable™, 0.5 M—Packard BioScience Co. Catalogue No. 6NE9 100 (NEF910)). Dilutions for creating a standard curve were also prepared containing from 0-250 μg of melanin (Sigma cat. M 8631) in a total of 250 μl Solvable™. The tissues were incubated in their tubes overnight at about 60° C. overnight along with the dilutions. The tissue samples were then vortexed. Then the tissue samples were centrifuged at 13,000 rpm for 5 minutes to pellet. The supernatant from the tissue samples was collected. 250 μl of the supernatant from each tissue sample as well as the dilutions were then added to separate wells of a microwell plate. The plate was read at 490 nM. The optical densities of the dilutions were used to prepare a standard curve against which the optical densities of the tissue sample supernatants could be compared to determine the melanin content of the tissue samples.

Skin Lightening

The fixed tissues for Days 3, 7, 10, and 14 were observed macroscopically. Comparing the tissues over time showed that the tissues darken progressively with increased time in culture for all test articles. As expected, at Days 3, 7, and 10 the positive control (KA) was lighter than the negative control (water). There was also a lightening effect produced by test articles SB, A, FS, DG, and Mix as compared with the negative control that was visible at all time points. Tissue samples treated with GS, however, did not appear distinguishably lighter or darker than the negative control tissue samples.

Melanin Assay

Melanin assay results at Days 10 and 14 are shown in Table 2. As expected, the melanin content of tissue samples treated with the negative control (H₂O) increased from 24.6 μg/tissue at Day 10 to 37.1 μg/tissue at Day 14. Tissues treated with the positive control (kojic acid) as well as those treated with GS, SB, A, and the Mix all showed lower melanin content than the negative control on Day 10 and Day 14. Treatment with test article A resulted in the largest decrease in melanin content as compared to the negative control and resulted in greater inhibition than the positive control at both time points. The Mix resulted in the next largest decrease in melanin content at Day 10, and the third largest decrease in melanin content behind test article A and the positive control at Day 14 (as compared with the negative control). However, when the melanin assay data and cell viability data are compared, the Mix is the most efficacious of the test articles in inhibiting melanin production.

In this regards, although test article A caused the greatest reduction in melanin production, it also caused the greatest reduction in cell viability as compared to the negative control, as noted above and shown in Table 1. Each other test article also decreased cell viability relative to the Mix at one or more of the tested amounts. Thus, test article A and the other test articles were acting on fewer cells than the Mix. The Mix, on the other hand, concurrently increased cell viability at each amount tested and inhibited melanin production (as compared to the negative control). The inhibition caused by the Mix was greater than each test article other than test article A. Thus, the Mix is the most efficacious of the articles tested in inhibiting melanin production.

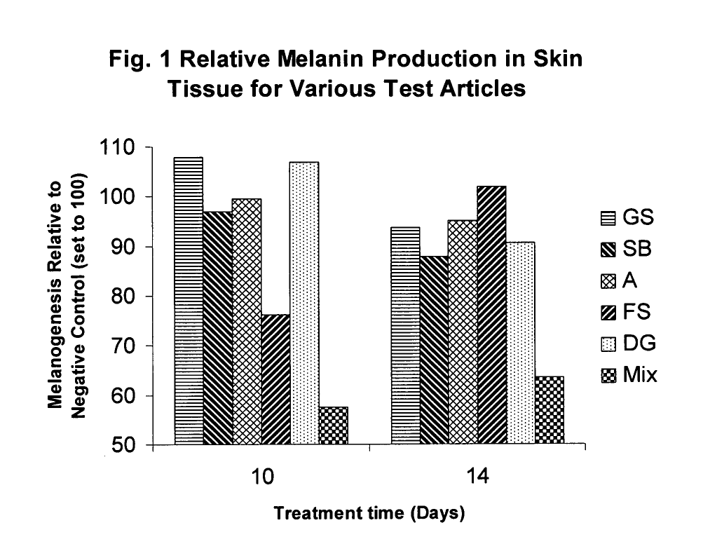

The efficacy of the Mix is shown graphically in FIG. 1. The data in FIG. 1 were derived by extrapolating the 48-hour cell viability data discussed above to estimate the cellular growth of the Epiderm tissues over an additional 12 days. The growth of the tissue exposed to the negative control was used as the standard and a projected growth curve was generated. Using the cell viability data obtained for the other test articles, projected growth curves for the tissue samples as exposed to the other test articles were generated. The average melanin content from Table 2 for each of the test articles was then divided by the projected cellular growth at Days 10 and 14 for the tissue exposed to the corresponding test article. The result for the negative control was set at 100 for both days 10 and 14 and the results for the other test articles were appropriately adjusted. FIG. 1 shows the melanogenesis in the tissues exposed to the various test articles relative to the melanogeneis in the tissues exposed to the negative control, thus also demonstrating the relative efficacy of the various test articles in inhibiting melanogensis. When the relative efficacy of the test articles is examined, the Mix is dramatically more efficacious than any of the individual ingredients in the Mix.

TABLE 2

| | Melanin | | | % | % |
|---|---|---|---|---|---|
| | content of the | | | melanin | melanin |
| | two tested | | | content | content |
| | samples for | Average | | relative | relative |
| | each test | melanin | | to the | to the |
| | article | content | Standard | Negative | Positive |
| Test article or control | (μg/tissue) | (μg/tissue) | Deviation | Control | Control |
| Negative Control Day 10 | 25.4 | 24.6 | 1.01 | 100.0 | 108.8 |
| | 23.9 | | | | |
| Negative Control Day 14 | 33.8 | 37.1 | 4.62 | 100.0 | 138.3 |
| | 40.3 | | | | |
| Positive Control Day 10 | 25.0 | 22.7 | 3.38 | 91.9 | 100.0 |
| | 20.3 | | | | |
| Positive Control Day 14 | 26.6 | 26.8 | 0.23 | 72.3 | 100.0 |
| | 27.0 | | | | |
| G.S. Day 10 | 21.7 | 23.6 | 2.70 | 95.8 | 104.2 |
| | 25.5 | | | | |
| G.S. Day 14 | 28.2 | 30.9 | 3.72 | 83.2 | 115.2 |
| | 33.5 | | | | |
| S.B. Day 10 | 25.4 | 22.7 | 3.83 | 91.9 | 100.0 |
| | 19.9 | | | | |
| S.B. Day 14 | 32.5 | 31.0 | 6.90 | 83.7 | 115.8 |
| | 29.5 | | | | |
| A. Day 10 | 18.5 | 21.5 | 4.28 | 87.4 | 95.1 |
| | 24.6 | | | | |
| A. Day 14 | 29.7 | 22.2 | 10.59 | 59.8 | 82.8 |
| | 14.7 | | | | |
| F.S. Day 10 | 21.1 | 20.9 | 0.23 | 84.8 | 92.3 |
| | 20.7 | | | | |
| F.S. Day 14 | 36.7 | 41.4 | 6.76 | 111.8 | 154.7 |
| | 46.2 | | | | |
| D.G. Day 10 | 27.3 | 24.8 | 3.49 | 100.6 | 109.5 |
| | 22.3 | | | | |
| D.G. Day 14 | 26.0 | 31.8 | 8.22 | 85.8 | 118.7 |
| | 37.6 | | | | |
| MIX Day 10 | 20.4 | 17.6 | 4.06 | 71.2 | 77.5 |
| | 14.7 | | | | |
| MIX Day 14 | 26.8 | 29.3 | 3.49 | 78.9 | 109.2 |
| | 31.7 | | | | |

Melanocyte Morphology and Dendricity

The samples of the tissues treated with each test article and control fixed on Days 3, 7, 10, and 14, were observed microscopically to examine melanocyte morphology and dendricity. Each sample observed was essentially normal.

Tissue Morphology

The samples of the tissues treated with each test article and control fixed on Days 3, 7, 10, and 14 were cross sectioned and stained with hematoxylin and eosin so that histological chances could be observed. The stratum corneum of the tissue samples thickened for all the treatments when earlier fixed samples were compared against later fixed samples. Otherwise, all the treated tissues retained essentially normal epidermal structure over the course of the study.

### EXAMPLE 2

This is an example of an exemplary embodiment of the disclosed topical composition comprising the disclosed melanogenesis inhibiting formulation and a dermatologically acceptable carrier in the form of a cream, as well as other ingredients, such as additional skin lighteners, for example, Morus alba extract (white mulberry). The disclosed melanogenesis inhibiting formulation in this embodiment comprises about 3.2% of the total composition by weight. The dermatologically acceptable carrier comprises among other ingredients, water, glycerin, pentylene glycol, C12-15 alkyl benzoate, caprylic/capric triglyceride, and dimethicone.

Cream

| Ingredient | % by weight |
|---|---|
| water | 66.322 |
| cyclopentasiloxane | 6.000 |
| glycerin | 3.000 |
| pentylene glycol | 3.000 |
| ascorbyl glucoside | 2.000 |
| C12-15 alkyl benzoate | 3.000 |
| caprylic/capric triglyceride | 2.500 |
| Fagus sylvatica extract | 2.000 |
| dimethicone | 1.000 |
| myristal myristate | 3.000 |
| Arctostaphylos uva ursi leaf extract | 0.500 |
| dipotassium glycyrrhizate | 0.100 |
| Glycine soja seed extract | 0.500 |
| Scutellaria baicalensis root extract | 0.100 |
| algae extract | 0.050 |
| Morus alba root extract | 0.050 |
| Rosmarinus officinalis leaf extract | 0.050 |
| Saxifraga sarmentosa extract | 0.050 |
| Uncaria gambir extract | 0.010 |
| Aloe barbadensis leaf extract | 0.015 |
| Chlorella vulgaris extract | 0.015 |
| Pueraria lobata root extract | 0.015 |
| Althaea officinalis root extract | 0.012 |
| Arnica montana extract | 0.012 |
| Paeonia suffruticosa root extract | 0.012 |
| Tilia cordata flower extract | 0.012 |
| tocopherol | 0.100 |
| creatin | 0.050 |
| sodium hyaluronate | 0.100 |
| Arachis hypogaea oil | 0.025 |
| ceramide 3 | 0.0025 |
| sodium PCA | 0.100 |
| cholestrol | 0.100 |
| glyceryl stearate SE | 0.750 |
| potassium hydroxide | 0.700 |
| hydrogenated lecithin | 0.500 |
| magnesium aluminum silicate | 0.450 |
| acrylates/C10-30 alkylacrylate crosspolymer | 0.400 |
| carbomer | 0.400 |
| PEG-20 sorbitan cocoate | 0.300 |
| PEG-6 | 0.300 |
| PEG-8 | 0.300 |
| phenoxyethanol | 0.300 |
| steareth-20 | 0.300 |
| glycosyl trehalose | 0.200 |
| hydrogenated starch hydrolysate | 0.200 |
| butylene glycol | 0.150 |
| pentasodium pentetate | 0.100 |
| xylitol | 0.050 |
| retinyl palmitate | 0.025 |
| sodium metabisulfite | 0.150 |
| maltitol | 0.015 |
| behenyl alcohol | 0.0025 |
| polyglyceryl-10 pentastearate | 0.0025 |
| sodium steroyl lactylate | 0.0025 |
| phenoxyethanol | 0.300 |
| sodium gluconate | 0.300 |

### EXAMPLE 3

This is an example of an exemplary embodiment of the disclosed topical composition comprising the disclosed melanogenesis inhibiting formulation and a dermatologically acceptable carrier in the form of a day lotion milk, as well as other ingredients, such as additional skin lighteners, for example, Morus alba extract (white mulberry). The disclosed melanogenesis inhibiting formulation in this embodiment comprises about 3.2% of the total composition by weight. The dermatologically acceptable carrier comprises among other ingredients, water, glycerin, pentylene glycol, butylene glycol, caprylic/capric triglyceride, and polyglyceryl-10-stearate.

Day Lotion Milk

| Ingredient | % by weight |
|---|---|
| water | 60.645 |
| octyldodecyl neopentanoate | 4.000 |
| ethylhexyl methoxycinnamate | 5.000 |
| caprylic/capric triglyceride | 3.000 |
| cyclopentasiloxane | 5.000 |
| glycerin | 5.000 |
| pentylene glycol | 2.000 |
| ascorbyl glucoside | 2.000 |
| Fagus sylvatica extract | 2.000 |
| butyl methoxydibenzoylmethane | 1.500 |
| cetyl palmitate | 1.500 |
| glycosyl trehalose | 0.500 |
| hydrogenated starch hydrolysate | 0.500 |
| lauroyl lysine | 0.500 |
| Arctostaphylos uva ursi leaf extract | 0.500 |
| dipotassium glycyrrhizate | 0.100 |
| Glycine soja seed extract | 0.500 |
| Scutellaria baicalensis root extract | 0.100 |
| algae extract | 0.050 |
| Morus alba root extract | 0.050 |
| Rosmarinus officinalis leaf extract | 0.050 |
| Saxifraga sarmentosa extract | 0.050 |
| Uncaria gambir extract | 0.010 |
| Aloe barbadensis leaf extract | 0.015 |
| Chlorella vulgaris extract | 0.015 |
| Pueraria lobata root extract | 0.015 |
| Althaea officinalis root extract | 0.012 |
| Arnica montana extract | 0.012 |
| Paeonia suffruticosa root extract | 0.012 |
| Tilia cordata flower extract | 0.012 |
| tocopherol | 0.01000 |
| creatin | 0.05000 |
| sodium hyaluronate | 0.100 |
| Arachis hypogaea oil | 0.0005 |
| Simmondsia chinensis seed oil | 0.500 |
| squalane | 0.500 |
| ceramide 3 | 0.00025 |
| triethanolamine | 0.800 |
| polyglyceryl-10-stearate | 1.000 |
| potassium hydroxide | 0.400 |
| phenoxyethanol | 0.300 |
| acrylates/C10-30 alkylacrylate crosspolymer | 0.200 |
| carbomer | 0.200 |
| hydroxypropylcellulose | 0.100 |
| pentasodium pentetate | 0.05000 |
| disodium EDTA | 0.02000 |
| sodium metabisulfite | 0.02000 |
| retinyl palmitate | 0.00050 |
| behenyl alcohol | 0.00025 |
| polyglyceryl-10 pentastearate | 0.00025 |
| sodium stearoyl lactylate | 0.00025 |
| butylene glycol | 1.100 |

### EXAMPLE 4

This is an example of an exemplary embodiment of the disclosed topical composition comprising the disclosed melanogenesis inhibiting formulation and a dermatologically acceptable carrier in the form of an essence preparation, as well as other ingredients, such as additional skin lighteners, for example, Morus alba extract (white mulberry). The disclosed melanogenesis inhibiting formulation in this embodiment comprises about 3.2% of the total composition by weight. The dermatologically acceptable carrier comprises among other ingredients, water, glycerin, pentylene glycol, butylene glycol, and PEG-11 methyl ether dimethicone.

Essence

| Ingredient | % by weight |
|---|---|
| water | 75.832 |
| butylene glycol | 5.500 |
| pentylene glycol | 2.500 |
| glycerin | 4.000 |
| PEG-11 methyl ether dimethicone | 2.000 |
| sodium hyaluronate | 1.000 |
| ascorbyl glucoside | 2.000 |
| Fagus sylvatica extract | 2.000 |
| Rosmarinus officinalis leaf extract | 1.000 |
| Arctostaphylos uva ursi leaf extract | 0.500 |
| dipotassium glycyrrhizate | 0.100 |
| Glycine soja seed extract | 0.500 |
| Scutellaria baicalensis root extract | 0.100 |
| algae extract | 0.250 |
| Morus alba root extract | 0.050 |
| Saxifraga sarmentosa extract | 0.050 |
| Uncaria gambir extract | 0.010 |
| Aloe barbadensis leaf extract | 0.100 |
| Chlorella vulgaris extract | 0.100 |
| Pueraria lobata root extract | 0.100 |
| Althaea officinalis root extract | 0.012 |
| Arnica montana extract | 0.012 |
| Paeonia suffruticosa root extract | 0.012 |
| Tilia cordata flower extract | 0.012 |
| tocopherol | 0.010 |
| creatine | 0.050 |
| glycosyl trehalose | 0.050 |
| potassium hydroxide | 0.600 |
| acrylates/C10-30 alkylacrylate crosspolymer | 0.400 |
| phenoxyethanol | 0.300 |
| styrene/PVP copolymer | 0.200 |
| PEG-50 castor oil | 0.060 |
| hydrogenated starch hydrolysate | 0.050 |
| pentasodium pentetate | 0.050 |
| polysorbate 20 | 0.040 |
| sodium metabisulfite | 0.150 |
| sodium gluconate | 0.300 |

### EXAMPLE 5

This is an example of an exemplary embodiment of the disclosed topical composition comprising the disclosed melanogenesis inhibiting formulation and a dermatologically acceptable carrier in the form of a toner/lotion, as well as other ingredients, such as additional skin lighteners, for example, Morus alba extract (white mulberry). The disclosed melanogenesis inhibiting formulation in this embodiment comprises about 0.36% of the total composition by weight. The dermatologically acceptable carrier comprises among other ingredients, water, glycerin, pentylene glycol, butylene glycol, and PEG-10 methyl ether dimethicone.

Toner/Lotion

| Ingredient | % by weight |
|---|---|
| water | 80.530 |
| butylene glycol | 6.000 |
| glycerin | 3.000 |
| pentylene glycol | 2.000 |
| PEG-10 methyl ether dimethicone | 3.000 |
| ascorbyl glucoside | 2.000 |
| Fagus sylvatica extract | 0.200 |
| Arctostaphylos uva ursi leaf extract | 0.050 |
| dipotassium glycyrrhizate | 0.050 |
| Glycine soja seed extract | 0.050 |
| Scutellaria baicalensis root extract | 0.010 |
| algae extract | 0.050 |
| Morus alba root extract | 0.050 |
| Rosmarinus officinalis leaf extract | 0.500 |
| Saxifraga sarmentosa extract | 0.050 |
| Uncaria gambir extract | 0.010 |
| Aloe barbadensis leaf extract | 0.015 |
| Chlorella vulgaris extract | 0.015 |
| Pueraria lobata root extract | 0.015 |
| Althaea officinalis root extract | 0.012 |
| Arnica montana extract | 0.012 |
| Paeonia suffruticosa root extract | 0.012 |
| Tilia cordata flower extract | 0.012 |
| gocopherol | 0.010 |
| sodium hyaluronate | 0.050 |
| glycosyl trehalose | 0.300 |
| hydrogenated starch hydrolysate | 0.300 |
| pentasodium pentetate | 0.100 |
| sodium gluconate | 0.300 |
| 1-methylhydantoin-2-imide | 0.050 |
| PEG-50 castor oil | 0.060 |
| potassium hydroxide | 0.377 |
| phenoxyethanol | 0.300 |
| polysorbate 20 | 0.040 |
| styrene/PVP copolymer | 0.020 |
| sodium metabisulfite | 0.150 |
| sodium citrate | 0.200 |
| citric acid | 0.100 |

### EXAMPLE 6

This is an example of an exemplary embodiment of the disclosed topical composition comprising the disclosed melanogenesis inhibiting formulation and a dermatologically acceptable carrier in the form of a cleansing foam, as well as other ingredients, such as additional skin lighteners, for example, Morus alba extract (white mulberry). The disclosed melanogenesis inhibiting formulation in this embodiment comprises about 0.41% of the total composition by weight. The dermatologically acceptable carrier comprises among other ingredients, water, glycerin, PEG-20, and butylene glycol.

Cleansing Foam

| Ingredient | % by weight |
|---|---|
| water | 25.583 |
| PEG-20 | 19.000 |
| myristic acid | 13.500 |
| stearic acid | 15.000 |
| glycerin | 9.000 |
| potassium myristoyl glutamate | 6.500 |
| potassium hydroxide | 6.250 |
| glycosyl trehalose | 1.000 |
| glycol distearate | 1.000 |
| Fagus sylvatica extract | 0.200 |
| Arctostaphylos uva ursi leaf extract | 0.050 |
| dipotassium glycyrrhizate | 0.100 |
| Glycine soja seed extract | 0.050 |
| Scutellaria baicalensis root extract | 0.010 |
| algae extract | 0.010 |
| Morus alba root extract | 0.010 |
| Rosmarinus officinalis leaf extract | 0.900 |
| Saxifraga sarmentosa extract | 0.010 |
| Uncaria gambir extract | 0.010 |
| Aloe barbadensis leaf extract | 0.003 |
| Chlorella vulgaris extract | 0.003 |
| Pueraria lobata root extract | 0.003 |
| Althaea officinalis root extract | 0.002 |
| Arnica montana extract | 0.002 |
| Paeonia suffruticosa root extract | 0.002 |
| Tilia cordata flower extract | 0.002 |
| tocopherol | 0.1000 |
| sodium hyaluronate | 0.1000 |
| Olea europaea fruit Oil | 0.5000 |
| hydrogenated starch hydrolysate | 0.700 |
| butylene glycol | 0.1000 |
| phenoxyethanol | 0.3000 |

### EXAMPLE 7

This is an example of an exemplary embodiment of the disclosed topical composition comprising the disclosed melanogenesis inhibiting formulation and a dermatologically acceptable carrier in the form of a cleansing gel, as well as other ingredients, such as additional skin lighteners, for example, Morus alba extract (white mulberry). The disclosed melanogenesis inhibiting formulation in this embodiment comprises about 0.41% of the total composition by weight. The dermatologically acceptable carrier comprises among other ingredients, water, glycerin, PEG-20, and butylene glycol.

Cleansing Gel

| Ingredient | % by weight |
|---|---|
| water | 80.083 |
| butylene glycol | 10.000 |
| PEG-7 caprylic/capric glyceride | 3.000 |
| lauryl glucoside | 2.500 |
| glycerin | 1.000 |
| carbomer | 1.000 |
| Fagus sylvatica extract | 0.200 |
| Arctostaphylos uva ursi leaf extract | 0.050 |
| dipotassium glycyrrhizate | 0.100 |
| Glycine soja seed extract | 0.050 |
| Scutellaria baicalensis root extract | 0.010 |
| algae extract | 0.010 |
| Morus alba root extract | 0.010 |
| Rosmarinus officinalis leaf extract | 0.900 |
| Saxifraga sarmentosa extract | 0.010 |
| Uncaria gambir extract | 0.010 |
| Aloe barbadensis leaf extract | 0.003 |
| Chlorella vulgaris extract | 0.003 |
| Pueraria lobata root extract | 0.003 |
| Althaea officinalis root extract | 0.002 |
| Arnica montana extract | 0.002 |
| Paeonia suffruticosa root extract | 0.002 |
| Tilia cordata flower extract | 0.002 |
| Borago officinalis seed oil | 0.100 |
| Simmondsia chinensis seed oil | 0.100 |
| pentasodium pentetate | 0.050 |
| squalane | 0.050 |
| potassium hydroxide | 0.450 |
| phenoxyethanol | 0.300 |

### EXAMPLE 8

This is an example of an exemplary embodiment of a method of lightening a subject's skin using the disclosed topical composition. A topical composition is provided comprising about 3.2% of the disclosed melanogenesis inhibiting formulation, wherein the melanogenesis inhibiting formulation comprises about 15% Arctostaphylos uva ursi extract, about 3% Scutellaria baicalensis extract, about 3% glycyrrhizic acid, about 15% Glycine soja extract, and about 63% Fagus sylvatica extract (all % by weight), and further comprising a dermatologically acceptable carrier in the form of a cream comprising water, glycerin, pentylene glycol, C12-15 alkyl benzoate, caprylic/capric triglyceride, and dimethicone. The topical composition is applied to an area of a subject skin in the amount of about 2 mg of the topical composition per cm² of the area of skin to be lightened. A similar dosage amount is applied each day until the subject is satisfied with the degree of lightening in the treated area of his or her skin. In this example, the dosage is applied for 10 days at which time the treated area of the subject skin is of about the same color as an adjacent non-treated area that was initially lighter in color than the treated area.

### EXAMPLE 9

This Example provides the results of a human trial of the skin lightening effects of the disclosed topical composition and demonstrates the effectiveness of the disclosed topical composition on hyperpigmented human skin.

Thirty-one participants completed the trial (all females of Asian descent). The participants in the trial were instructed to follow a regimen of applying to a hyperpigmented area of their skin and the surrounding skin a variety of embodiments of the disclosed topical composition for twelve weeks. The regimen comprised applying to an area of skin including a hyperpigmented area the following products all comprising the disclosed topical composition:
A. During the day, in the following order: 1) Cleansing Foam; 2) Toner; 3) Essence; 4) Spot Treatment; 5) Day Lotion Milk; and
B. At night, in the following order: 1) Cleansing Gel; 2) Cleansing Foam; 3) Toner; 4) Essence; 5) Spot Treatment; 6) Cream.

The compositions of the various products comprising the products used in the trial are disclosed in Example 10.

Participants were instructed to abstain from using any skin lightening products (topical or oral) and to avoid direct daily sun exposure on the face, neck and arms for at seven days prior to commencement of the trial commencing and to also avoid the sun and not use any skin lightening products other than those provided by the trial during the course of the trial.

Chromametry analysis was performed on the hyperpigmented area of each participant's skin and the surrounding skin at baseline (Day 0) and at Weeks 4, 8, and 12 using a Spectrophotometer CM-2600d. This instrument utilizes the D/8 geometry conforming to CIE No. 15, ISO 7724/1, ASTM E1164, DIN 5033 Tei17, and JIS Z8722-1982 (diffused illumination/8° viewing system) standards, and offers simultaneous SCI (Specular Component Included) and SCE (Specular Component Excluded) measurements. Light from Xenon lamps diffuses on the inner surface of the integrating sphere and illuminates the specimen uniformly. The light reflected by the specimen surface at an angle of 8 degrees to the normal of the surface is received by the specimen-measuring optical system. The diffused light in the integrating sphere is received by the illumination-monitoring optical system and guided to the sensor. The light reflected by the specimen surface and the diffused light are divided into each wavelength component by the specimen-measuring optical system and illumination-monitoring optical sensor, respectively, and then signal proportional to the light intensity of each component is output to the analog processing circuit. By using the outputs from the specimen-measuring optical system and the illumination-monitoring sensor for calculation, compensation for slight fluctuation in spectral characteristics and the intensity of the illumination light is performed. (Double-beam system).

Skin color measurements were assessed with the L*a*b* system in which L* (white/black axis) is the quantity of reflected light or skin brightness ranging from total black (L*=0) to total white (L*=100), whereas b* (blue/yellow axis) is indicative of pigmentation (yellowness) and a* (red/green axis) reflects the degree of skin redness. In order to quantify skin pigmentation, the skin tan value ITA° [ITA°=arc tangent (L*−50/b*)×180/π] was calculated. The ITA° value is inversely correlated to skin pigmentation; thus, increases in measured ITA° values indicate a skin lightening effect.

Tables 3 and 4 provide the results of the measurements of the colorimetric changes of the participants' hyperpigmented areas of skin and the surrounding skin from baseline to week 12.

TABLE 3
Colorimetric changes of hyperpigmented areas from baseline to week 12.

| | | ITA° (mean) | |
|---|---|---|---|
| | ITA° (mean) | Difference from | Percentage |
| Assessment | Hyperpigmented Area | Baseline | Increase |
| Baseline | 15.81 ± 10.78 (week 4) | 0 | |
| (Day 0)* | 16.48 ± 11.15 (week 8) | | |
| | 17.35 ± 12.08 (week 12) | | |
| 4 weeks | 20.34 ± 11.16 | 4.53 | 28.7 |
| 8 weeks | 23.22 ± 10.89 | 6.74 | 40.9 |
| 12 weeks | 29.24 ± 14.99 | 11.89 | 68.5 |

*Baseline means differ week to week to account for partial completion of the trial by certain participants (with results for participants not evaluated in subsequent weeks being excluded from the baseline mean calculation for that week).

TABLE 4
Colorimetric changes of surrounding skin areas
from baseline to Week 12.

| | | ITA° (mean) | |
|---|---|---|---|
| | ITA° (mean) | Difference from | Percentage |
| Assessment | Surrounding Skin | Baseline | Increase |
| Baseline | 31.18 ± 8.05 (week 4) | 0 | |
| (Day 0)* | 31.01 ± 8.23 (week 8) | | |
| | 32.06 ± 8.81 (week 12) | | |
| 4 weeks | 32.67 ± 1.4 | 1.49 | 4.8 |
| 8 weeks | 34.07 ± 6.15 | 3.06 | 9.8 |
| 12 weeks | 35.64 ± 5.53 | 3.58 | 11.2 |

*Baseline means differ week to week to account for partial completion of the trial by certain participants (with results for participants not evaluated in subsequent weeks being excluded from the baseline mean calculation for that week).

As can be seen in Tables 3-4, the calorimetric measurements of skin tan values (ITA°) changed over the course of the treatment period. On the areas of normal skin surrounding the hyperpigmented areas, ITA° values increased only slightly from about 32.06 at baseline to approximately 35.64 during the 12-week trial period (a total increase of about 3.58 or 11.2%). On the hyperpigmented areas, ITA° increased from about 15.1 at baseline to approximately 29.24 after 12 weeks (a total increase of about 11.89 or 68.5%), indicating lightening of the skin. Also, the difference between the hyperpigmented area and the surrounding skin at the baseline was about 15. By week 12 the difference was only about 6.

These results demonstrate that the disclosed topical composition is effective in lightening a subject's skin, particularly hyperpigmented areas of skin, as well as creating a more even-toned skin pigmentation between initially hyperpigmented areas and the surrounding skin.

### EXAMPLES 10(A)-(G)

Examples 10(a)-(g) disclose the embodiments of the disclosed topical composition used in the trial disclosed in Example 9.

### EXAMPLE 10(A)

The following is an exemplary embodiment of the disclosed topical composition comprising the disclosed melanogenesis inhibiting formulation and a dermatologically acceptable carrier in the form of a spot treatment, as well as other ingredients, such as additional skin lighteners, for example, ascorbyl glucoside. The disclosed melanogenesis inhibiting formulation in this embodiment comprises about 2.24% of the total composition by weight and the Scutellaria extract is a Scutellaria galericulata extract. The dermatologically acceptable carrier comprises among other ingredients, water, glycerin, and butylene glycol.

Spot Treatment

| | % by |
|---|---|
| Ingredient | weight |
| Water | 73.855 |
| Gransil ™ SiW-26 - a formulation of cyclopentasiloxane, | 10.000 |
| polysilicone-11, water, dimethicone and butylene glycol | |
| Sepiplus ™ 400 - a formulation of polyacrylate-13 and | 3.000 |
| polyisobutene and polysorbate 20 | |
| ascorbyl glucoside | 2.000 |
| Fagus sylvatica (beech tree Bud) extract | 1.000 |
| Arctostaphylos uva ursi (bear berry) leaf extract (Melfade J.) | 1.000 |
| tocopheryl acetate | 1.000 |
| sodium hyaluronate 1% solution. | 1.000 |
| Symglucan ™- a formulation of water, glycerin and beta | 1.000 |
| glucan | |
| molasses extract (molasses liquid) (Dr. | 1.000 |
| Straetman/Kientik ™) | |
| dextran (and) hexapeptide-2 (dermostatyl-dm) | 1.000 |
| Symdiol ™ 68T - a formulation of 1,2 hexane diol, 1,2 | 1.000 |
| octane diol, and tropolone | |
| sodium polyacrylate (Cosmedia ™ SP) | 1.000 |
| a water, butylene glycol, Saxifraga sarmentosa extract, | 0.5000 |
| Paeonia suffructicosa root extract, Pueraria lobata root | |
| extract formulation (Cellule Blanc ™) | |
| butylene glycol | 0.39500 |
| tetrasodium EDTA | 0.300 |
| sodium metabisulphite | 0.250 |
| NaOH | 0.200 |
| Glycine soja (soybean) seed extract | 0.11500 |
| Chlorella vulgaris (green algae) extract | 0.1000 |
| polysorbate 20 (Tween ™ 20) | 0.100 |
| dipotassium glycyrrhizate | 0.100 |
| Scutellaria galericulata (scull cap) root extract | 0.02460 |
| algae extract | 0.01230 |
| Morus alba (mulberry) root extract | 0.01230 |
| Rosmarinus officinalis (rosemary) leaf extract | 0.01230 |
| Aloe barbadensis leaf extract | 0.00420 |
| Pueraria lobata root extract | 0.00420 |
| Althae officinalis root extract | 0.00310 |
| Arnica montana flower extract | 0.00310 |
| Paeonia albiflora root extract | 0.00310 |
| Tilia cordata flower extract | 0.00310 |
| Uncaria tomentosa (cat's claw) extract | 0.00250 |

### EXAMPLE 10(B)

The following is an exemplary embodiment of the disclosed topical composition comprising the disclosed melanogenesis inhibiting formulation and a dermatologically acceptable carrier in the form of a cream, as well as other ingredients, such as additional skin lighteners, for example, Morus alba extract (white mulberry). The disclosed melanogenesis inhibiting formulation in this embodiment comprises about 3.2% of the total composition by weight. The dermatologically acceptable carrier comprises among other ingredients, water, glycerin, pentylene glycol, C12-15 alkyl benzoate, caprylic/capric triglyceride, and dimethicone.

Cream

| Ingredient | % by weight |
|---|---|
| Water | 68.090 |
| cyclopentasiloxane | 5.000 |
| glycerin | 5.000 |
| pentylene glycol | 3.000 |
| ascorbyl glucoside | 2.000 |
| C12-15 alkyl benzoate | 2.000 |
| caprylic/capric triglyceride | 2.000 |
| Fagus sylvatica (beach tree bud) extract | 2.000 |
| dimethicone | 1.500 |
| myristal myristate | 1.500 |
| Arctostaphylos uva ursi (bear berry) leaf extract | 0.500 |
| dipotassium glycyrrhizate | 0.100 |
| Glycine soja (soybean) seed extract | 0.500 |
| Scutellaria baicalensis (scull cap) root extract | 0.100 |
| algae extract | 0.050 |
| Morus alba (mulberry) root extract | 0.050 |
| Rosmarinus officinalis (rosemary) leaf extract | 0.050 |
| Saxifraga sarmentosa (strawberry geranium) extract | 0.050 |
| Uncaria gambir (cat's claw) extract | 0.01000 |
| Aloe barbadensis leaf extract | 0.01667 |
| Chlorella vulgaris (green algae) extract | 0.01667 |
| Pueraria lobata (kudzo) root extract | 0.01667 |
| Althaea officinalis (marshmallow) root extract | 0.01250 |
| Arnica montana extract | 0.01250 |
| Paeonia suffruticosa (peoney) root extract | 0.01250 |
| Tilia cordata (linden) flower extract | 0.01250 |
| tocopherol | 0.100 |
| creatin | 0.050 |
| sodium hyaluronate | 0.100 |
| Arachis hypogaea (peanut) oil | 0.02500 |
| ceramide 3 | 0.002500 |
| sodium PCA | 0.150 |
| cholestrol | 0.100 |
| glyceryl stearate SE | 0.800 |
| potassium hydroxide | 0.725 |
| hydrogenated lecithin | 0.500 |
| magnesium aluminum silicate | 0.450 |
| acrylates/C10-30 alkylacrylate crosspolymer | 0.400 |
| carbomer | 0.400 |
| PEG-20 sorbitan cocoate | 0.300 |
| PEG-6 | 0.300 |
| PEG-8 | 0.300 |
| phenoxyethanol | 0.300 |
| steareth-20 | 0.300 |
| butylene glycol | 0.150 |
| pentasodium pentetate | 0.100 |
| xylitol | 0.050 |
| retinyl palmitate | 0.02500 |
| sodium metabisulfite | 0.15000 |
| maltitol | 0.015 |
| behenyl alcohol | 0.002500 |
| polyglyceryl-10 pentastearate | 0.002500 |
| sodium steroyl lactylate | 0.002500 |
| phenoxyethanol | 0.300 |
| sodium gluconate | 0.300 |

### EXAMPLE 10(C)

The following is an exemplary embodiment of the disclosed topical composition comprising the disclosed melanogenesis inhibiting formulation and a dermatologically acceptable carrier in the form of a day lotion milk, as well as other ingredients, such as additional skin lighteners, for example, Morus alba extract (white mulberry). The disclosed melanogenesis inhibiting formulation in this embodiment comprises about 3.2% of the total composition by weight. The dermatologically acceptable carrier comprises among other ingredients, water, glycerin, PEG-20, and butylene glycol.

Day Lotion Milk

| Ingredient | % by weight |
|---|---|
| water | 63.858 |
| octyldodecyl neopentanoate | 5.000 |
| ethylhexyl methoxycinnamate | 4.200 |
| caprylic/capric triglyceride | 4.000 |
| cyclopentasiloxane | 3.500 |
| glycerin | 3.000 |
| pentylene glycol | 3.000 |
| ascorbyl glucoside | 2.000 |
| Fagus sylvatica (beach tree bud) extract | 2.000 |
| butyl methoxydibenzoylmethane | 1.400 |
| cetyl palmitate | 1.200 |
| lauroyl lysine | 1.000 |
| Arctostaphylos uva ursi (bear berry) leaf extract | 0.500 |
| dipotassium glycyrrhizate | 0.100 |
| Glycine soja (soybean) seed extract | 0.500 |
| Scutellaria baicalensis (scull cap) root extract | 0.100 |
| algae extract | 0.050 |
| Morus alba (mulberry) root extract | 0.050 |
| Rosmarinus officinalis (rosemary) leaf extract | 0.050 |
| Saxifraga sarmentosa (strawberry geranium) extract | 0.050 |
| Uncaria gambir (cat's claw) extract | 0.01000 |
| Aloe barbadensis leaf extract | 0.01667 |
| Chlorella vulgaris (green algae) extract | 0.01667 |
| Pueraria lobata (kudzo) root extract | 0.01667 |
| Althaea officinalis (marshmallow) root extract | 0.01250 |
| Arnica montana extract | 0.01250 |
| Paeonia suffruticosa (peoney) root extract | 0.01250 |
| Tilia cordata (linden) flower extract | 0.01250 |
| tocopherol | 0.01000 |
| creatin | 0.05000 |
| sodium hyaluronate | 0.100 |
| Arachis hypogaea (peanut) oil | 0.00050 |
| Simmondsia chinensis (jojoba) seed oil | 0.500 |
| squalane | 0.500 |
| ceramide 3 | 0.00025 |
| triethanolamine | 0.800 |
| polyglyceryl-10-stearate | 0.800 |
| potassium hydroxide | 0.380 |
| phenoxyethanol | 0.300 |
| acrylates/C10-30 alkylacrylate crosspolymer | 0.300 |
| carbomer | 0.300 |
| hydroxypropylcellulose | 0.100 |
| pentasodium pentetate | 0.05000 |
| disodium EDTA | 0.02000 |
| sodium metabisulfite | 0.02000 |
| retinyl palmitate | 0.00050 |
| behenyl alcohol | 0.00025 |
| polyglyceryl-10 pentastearate | 0.00025 |
| sodium stearoyl lactylate | 0.00025 |
| butylene glycol | 0.100 |

### EXAMPLE 10(D)

The following is an exemplary embodiment of the disclosed topical composition comprising the disclosed melanogenesis inhibiting formulation and a dermatologically acceptable carrier in the form of an essence preparation, as well as other ingredients, such as additional skin lighteners, for example, Morus alba extract (white mulberry). The disclosed melanogenesis inhibiting formulation in this embodiment comprises about 3.2% of the total composition by weight. The dermatologically acceptable carrier comprises among other ingredients, water, glycerin, pentylene glycol, butylene glycol, and PEG-11 methyl ether dimethicone.

Essence

| Ingredient | % by weight |
|---|---|
| water | 74.855 |
| butylene glycol | 5.000 |
| pentylene glycol | 4.000 |
| glycerin | 3.000 |
| PEG-11 methyl ether eimethicone | 2.000 |
| ascorbyl glucoside | 2.000 |
| Fagus sylvatica (beach tree bud) extract | 2.000 |
| Rosmarinus officinalis (rosemary) leaf extract | 1.000 |
| Arcostaphylos uva ursi (bear berry) leaf extract | 0.500 |
| dipotassium glycyrrhizate | 0.100 |
| Glycine soja (soybean) seed extract | 0.500 |
| Scutellaria baicalensis (scull cap) root extract | 0.100 |
| algae extract | 0.300 |
| Morus alba (mulberry) root extract | 0.050 |
| Saxifraga sarmentosa (strawberry geranium) extract | 0.050 |
| Uncaria gambir (cat's claw) extract | 0.01000 |
| Aloe barbadensis leaf extract | 0.100 |
| Chlorella vulgaris (green algae) extract | 0.100 |
| Pueraria lobata (kudzo) root extract | 0.100 |
| Althaea officinalis (marshmallow) root extract | 0.01250 |
| Arnica montana extract | 0.01250 |
| Paeonia suffruticosa (peoney) root extract | 0.01250 |
| Tilia cordata (linden) flower extract | 0.01250 |
| tocopherol | 0.01000 |
| creatine | 0.050 |
| sodium hyaluronate | 2.000 |
| potassium hydroxide | 0.625 |
| acrylates/C10-30 alkylacrylate crosspolymer | 0.400 |
| phenoxyethanol | 0.300 |
| styrene/PVP copolymer | 0.200 |
| PEG-50 castor oil | 0.060 |
| pentasodium pentetate | 0.050 |
| polysorbate 20 | 0.040 |
| sodium metabisulfite | 0.150 |
| sodium gluconate | 0.300 |

### EXAMPLE 10(E)

The following is an exemplary embodiment of the disclosed topical composition comprising the disclosed melanogenesis inhibiting formulation and a dermatologically acceptable carrier in the form of a toner/lotion, as well as other ingredients, such as additional skin lighteners, for example, Morus alba extract (white mulberry) and ascorbyl glucoside. The disclosed melanogenesis inhibiting formulation in this embodiment comprises about 0.36% of the total composition by weight. The dermatologically acceptable carrier comprises among other ingredients, water, glycerin, pentylene glycol, butylene glycol, and PEG-10 methyl ether dimethicone.

Toner

| Ingredient | % by weight |
|---|---|
| water | 80.430 |
| butylene glycol | 5.000 |
| glycerin | 4.000 |
| pentylene glycol | 4.000 |
| PEG-10 methyl ether dimethicone | 2.000 |
| ascorbyl glucoside | 2.000 |
| Fagus sylvatica (beach tree bud) extract | 0.200 |
| Arctostaphylos uva ursi (bear berry) leaf extract | 0.050 |
| dipotassium glycyrrhizate | 0.050 |
| Glycine soja (soybean) seed extract | 0.050 |
| Scutellaria baicalensis (scull cap) root extract | 0.010 |
| algae extract | 0.050 |
| Morus alba (mulberry) root extract | 0.050 |
| Rosmarinus officinalis (rosemary) leaf extract | 0.500 |
| Saxifraga sarmentosa (strawberry geranium) extract | 0.050 |
| Uncaria gambir (cat's claw) extract | 0.01000 |
| Aloe barbadensis leaf extract | 0.01667 |
| Chlorella vulgaris (green algae) extract | 0.01667 |
| Pueraria lobata (kudzo) root extract | 0.01667 |
| Althaea officinalis (marshmallow) root extract | 0.01250 |
| Arnica montana extract | 0.01250 |
| Paeonia suffruticosa (peoney) root extract | 0.01250 |
| Tilia cordata (linden) flower extract | 0.01250 |
| tocopherol | 0.01000 |
| sodium hyaluronate | 0.050 |
| pentasodium pentetate | 0.100 |
| sodium gluconate | 0.300 |
| 1-methylhydantoin-2-imide | 0.050 |
| PEG-50 castor oil | 0.060 |
| potassium hydroxide | 0.370 |
| phenoxyethanol | 0.300 |
| polysorbate 20 | 0.040 |
| styrene/PVP copolymer | 0.020 |
| sodium metabisulfite | 0.150 |
| sodium citrate (for pH adjustment) | QS |
| citric acid (for pH adjustment) | QS |

### EXAMPLE 10(F)

The following is an exemplary embodiment of the disclosed topical composition comprising the disclosed melanogenesis inhibiting formulation and a dermatologically acceptable carrier in the form of a cleansing foam, as well as other ingredients, such as additional skin lighteners, for example, Morus alba extract (white mulberry). The disclosed melanogenesis inhibiting formulation in this embodiment comprises about 0.41% of the total composition by weight. The dermatologically acceptable carrier comprises among other ingredients, water, glycerin, PEG-20, and butylene glycol.

Cleansing Foam

| Ingredient | % by weight |
|---|---|
| water | 25.030 |
| PEG-20 | 20.0000 |
| myristic acid | 14.0000 |
| stearic acid | 14.0000 |
| glycerin | 10.0000 |
| potassium myristoyl glutamate | 7.0000 |
| potassium hydroxide | 6.5000 |
| glycol distearate | 1.0000 |
| Fagus sylvatica (beach tree bud) extract | 0.200 |
| Arctostaphylos uva ursi (bear berry) leaf extract | 0.050 |
| dipotassium glycyrrhizate | 0.100 |
| Glycine soja (soybean) seed extract | 0.050 |
| Scutellaria baicalensis (scull cap) Root extract | 0.010 |
| algae extract | 0.0100 |
| Morus alba (mulberry) root extract | 0.0100 |
| Rosmarinus officinalis (rosemary) leaf extract | 0.9000 |
| Saxifraga sarmentosa (strawberry geranium) extract | 0.0100 |
| Uncaria gambir (cat's claw) extract | 0.0100 |
| Aloe barbadensis leaf extract | 0.0033 |
| Chlorella vulgaris (green algae) extract | 0.0033 |
| Pueraria lobata (kudzo) root extract | 0.0033 |
| Althaea officinalis (marshmallow) root extract | 0.0025 |
| Arnica montana extract | 0.0025 |
| Paeonia suffruticosa (peoney) root extract | 0.0025 |
| Tilia cordata (linden) flower extract | 0.0025 |
| tocopherol | 0.1000 |
| sodium hyaluronate | 0.1000 |
| Olea europaea (olive) fruit oil | 0.5000 |
| butylene glycol | 0.1000 |
| phenoxyethanol | 0.3000 |

### EXAMPLE 10(G)

The following is an exemplary embodiment of the disclosed topical composition comprising the disclosed melanogenesis inhibiting formulation and a dermatologically acceptable carrier in the form of a cleansing foam, as well as other ingredients, such as additional skin lighteners, for example, Morus alba extract (white mulberry). The disclosed melanogenesis inhibiting formulation in this embodiment comprises about 0.41% of the total composition by weight. The dermatologically acceptable carrier comprises among other ingredients, water, glycerin, PEG-20, and butylene glycol.

Cleansing Gel

| Ingredient | % by weight |
|---|---|
| water | 78.364 |
| butylene glycol | 12.000 |
| PEG-7 caprylic/capric glyceride | 4.000 |
| lauryl glucoside | 2.250 |
| carbomer | 1.000 |
| Fagus sylvatica (beach tree bud) extract | 0.200 |
| Arctostaphylos uva ursi (bear berry) leaf extract | 0.050 |
| dipotassium glycyrrhizate | 0.100 |
| Glycine soja (soybean) seed extract | 0.050 |
| Scutellaria baicalensis (scull cap) root extract | 0.010 |
| algae extract | 0.010 |
| Morus alba (mulberry) root extract | 0.010 |
| Rosmarinus officinalis (rosemary) leaf extract | 0.900 |
| Saxifraga sarmentosa (strawberry geranium) extract | 0.010 |
| Uncaria gambir (cat's claw) extract | 0.010 |
| Aloe barbadensis leaf extract | 0.00333 |
| Chlorella vulgaris (green algae) extract | 0.00333 |
| Pueraria lobata (kudzo) root extract | 0.00333 |
| Althaea officinalis (marshmallow) root extract | 0.00250 |
| Arnica montana extract | 0.00250 |
| Paeonia suffruticosa (peoney) root extract | 0.00250 |
| Tilia cordata (linden) flower extract | 0.00250 |
| Borago officinalis seed oil | 0.100 |
| Simmondsia chinensis (jojoba) seed oil | 0.100 |
| pentasodium pentetate | 0.050 |
| potassium hydroxide | 0.466 |
| phenoxyethanol | 0.300 |

In view of the many possible embodiments to which the principles of the disclosed invention may be applied, it should be recognized that the illustrated embodiments are only examples of the disclosed invention and should not be taken as limiting the scope of the invention. Rather, the scope of the invention is defined by the following claims. We therefore claim as our invention all that comes within the scope and spirit of these claims.

We claim:

1. A topical composition, comprising:
an Arctostaphylos uva ursi extract;
a Scutellaria extract;
a glycyrrhizic acid;
a Glycine soja extract; and
a Fagus sylvatica extract;
wherein the Arctostaphylos uva ursi extract, Scutellaria extract, glycyrrhizic acid, Glycine soja extract, and Fagus sylvatica extract are present in amounts effective to inhibit melanogenesis in skin tissue or lighten the color of skin tissue.

2. The topical composition of claim 1, further comprising a dermatologically acceptable carrier.

3. The topical composition of claim 2, wherein the Arctostaphylos uva ursi extract comprises a water extract from a leaf of an Arctostaphylos uva ursi plant.

4. The topical composition of claim 3, wherein the Scutellaria extract comprises a Scutellaria baicalensis extract.

5. The topical composition of claim 4, wherein the Scutellaria baicalensis extract comprises a water extract from a root of a Scutellaria baicalensis plant.

6. The topical composition of claim 5, wherein the glycyrrhizic acid comprises dipotassium glycyrrhizate.

7. The topical composition of claim 6, wherein the Glycine soja extract comprises a water extract from a seed of a Glycine soja plant.

8. The topical composition of claim 7, wherein the Fagus sylvatica extract comprises a water extract from a bud of a Fagus sylvatica plant.

9. The topical composition of claim 8, wherein the Arctostaphylos uva ursi extract comprises from 0.05% to 0.5% of the topical composition by weight, the Scutellaria baicalensis extract comprises from 0.01% to 0.2% of the topical composition by weight, the glycyrrhizic acid comprises from 0.05% to 0.2% of the topical composition by weight, the Glycine soja extract comprises from 0.05% to 0.5% of the topical composition by weight, and Fagus sylvatica extract comprises from 0.2% to 2% of the topical composition by weight.

10. The topical composition of claim 8, wherein the Arctostaphylos uva ursi extract, Scutellaria baicalensis extract, glycyrrhizic acid, Glycine soja extract, and Fagus sylvatica extract together comprise 0.35% to 3.2% of the topical composition by weight.

11. The topical composition of claim 8, wherein the Arctostaphylos uva ursi extract, Scutellaria baicalensis extract, glycyrrhizic acid, Glycine soja extract, and Fagus sylvatica extract together comprise 1% of the topical composition by weight.

12. The topical composition of claim 11, wherein the Arctostaphylos uva ursi extract, Scutellaria baicalensis extract, glycyrrhizic acid, Glycine soja extract, and Fagus sylvatica extract comprise equivalent percentages of the topical composition by weight.

13. The topical composition of claim 12, wherein the Arctostaphylos uva ursi extract comprises by weight 20% of the water extract from the leaf of the Arctostaphylos uva ursi plant, 39.5% water, 39.5% butylene glycol, and 1% of a stabilizing composition comprising paraben esters in phenoxyethanol; wherein the Scutellaria baicalensis extract comprises by weight 20% of the water extract from the root of the Scutellaria baicalensis plant, 39.5% water, 39.5% butylene glycol, and 1% of a stabilizing composition comprising paraben esters in phenoxyethanol; and wherein the Glycine soja extract comprises by weight 20% of the water extract from the seed of the Glycine soja plant, 39.5% water, 39.5% butylene glycol, and 1% of a stabilizing composition comprising paraben esters in phenoxyethanol.

14. The topical composition of claim 2 in the form of a formulation selected from the group consisting of:
A spot treatment, comprising,

| | % by weight |
|---|---|
| water | 73.855 |
| a cyclopentasiloxane, polysilicone-11, dimethicone, | 10.000 |
| butylene glycol, and water formulation | |
| a polyacrylate-13, polyisobutene, and polysorbate | 3.000 |
| 20 formulation | |
| ascorbyl glucoside | 2.000 |
| Fagus sylvatica extract | 1.000 |
| Arctostaphylos uva ursi leaf extract | 1.000 |
| tocopheryl acetate | 1.000 |
| sodium hyaluronate 1% soln. | 1.000 |
| a water, glycerin, and beta glucan formulation | 1.000 |
| molasses extract | 1.000 |
| dextran and hexapeptide-2 | 1.000 |
| a 1,2 hexane diol, 1,2 octane diol, and tropolone | 1.000 |
| formulation | |
| sodium polyacrylate | 1.000 |
| a water, butylene glycol, Saxifraga sarmentosa | 0.5000 |
| extract, Paeonia suffructicosa root extract, and | |
| Pueraria lobata root extract formulation | |
| butylene glycol | 0.39500 |
| tetrasodium EDTA | 0.300 |
| sodium metabisulphite | 0.250 |
| NaOH | 0.200 |
| Glycine soja extract | 0.11500 |
| Chlorella vulgaris extract | 0.1000 |
| polysorbate 20 | 0.100 |
| dipotassium glycyrrhizate | 0.100 |
| Scutellaria galericulata extract | 0.02460 |
| algae extract | 0.01230 |
| Morus alba root extract | 0.01230 |
| Rosmarinus officinalis leaf extract | 0.01230 |
| Aloe barbadensis leaf extract | 0.00420 |
| Pueraria lobata root extract | 0.00420 |
| Althae officinalis root extract | 0.00310 |
| Arnica montana flower extract | 0.00310 |
| Paeonia albiflora root extract | 0.00310 |
| Tilia cordata flower extract | 0.00310 |
| Uncaria tomentosa extract | 0.00250 |

a cream, comprising

| | % by weight |
|---|---|
| water | 68.090 |
| cyclopentasiloxane | 5.000 |
| glycerin | 5.000 |
| pentylene glycol | 3.000 |
| ascorbyl glucoside | 2.000 |
| C12-15 alkyl benzoate | 2.000 |
| caprylic/capric triglyceride | 2.000 |
| Fagus sylvatica extract | 2.000 |
| dimethicone | 1.500 |
| myristal myristate | 1.500 |
| Arctostaphylos uva ursi leaf extract | 0.500 |
| dipotassium glycyrrhizate | 0.100 |
| Glycine soja extract | 0.500 |
| Scutellaria baicalensis root extract | 0.100 |
| algae extract | 0.050 |
| Morus alba root extract | 0.050 |
| Rosmarinus officinalis leaf extract | 0.050 |
| Saxifraga sarmentosa extract | 0.050 |
| Uncaria gambir extract | 0.01000 |
| Aloe barbadensis leaf extract | 0.01667 |
| Chlorella vulgaris extract | 0.01667 |
| Pueraria lobata root extract | 0.01667 |
| Althaea officinalis root extract | 0.01250 |
| Arnica montana extract | 0.01250 |
| Paeonia suffruticosa root extract | 0.01250 |
| Tilia cordata flower extract | 0.01250 |
| gocopherol | 0.100 |
| creatin | 0.050 |
| sodium hyaluronate | 0.100 |
| Arachis hypogaea oil | 0.02500 |
| ceramide 3 | 0.002500 |
| sodium PCA | 0.150 |
| cholestrol | 0.100 |
| glyceryl stearate SE | 0.800 |
| potassium hydroxide | 0.725 |
| hydrogenated lecithin | 0.500 |
| magnesium aluminum silicate | 0.450 |
| acrylates/C10-30 alkylacrylate crosspolymer | 0.400 |
| carbomer | 0.400 |
| PEG-20 sorbitan cocoate | 0.300 |
| PEG-6 | 0.300 |
| PEG-8 | 0.300 |
| phenoxyethanol | 0.300 |
| steareth-20 | 0.300 |
| butylene glycol | 0.150 |
| pentasodium pentetate | 0.100 |
| xylitol | 0.050 |
| retinyl palmitate | 0.02500 |
| sodium metabisulfite | 0.15000 |
| maltitol | 0.015 |
| behenyl alcohol | 0.002500 |
| polyglyceryl-10 pentastearate | 0.002500 |
| sodium steroyl lactylate | 0.002500 |
| phenoxyethanol | 0.300 |
| sodium gluconate | 0.300 |

a day lotion milk comprising,

| | % by weight |
|---|---|
| water | 63.858 |
| octyldodecyl neopentanoate | 5.000 |
| ethylhexyl methoxycinnamate | 4.200 |
| caprylic/capric triglyceride | 4.000 |
| cyclopentasiloxane | 3.500 |
| glycerin | 3.000 |
| pentylene glycol | 3.000 |
| ascorbyl glucoside | 2.000 |
| Fagus sylvatica extract | 2.000 |
| Butyl methoxydibenzoylmethane | 1.400 |
| cetyl palmitate | 1.200 |
| lauroyl lysine | 1.000 |
| Arctostaphylos uva ursi leaf extract | 0.500 |
| dipotassium glycyrrhizate | 0.100 |
| Glycine soja extract | 0.500 |
| Scutellaria baicalensis root extract | 0.100 |
| algae extract | 0.050 |
| Morus alba root extract | 0.050 |
| Rosmarinus officinalis leaf extract | 0.050 |
| Saxifraga sarmentosa extract | 0.050 |
| Uncaria gambir extract | 0.01000 |
| Aloe barbadensis leaf extract | 0.01667 |
| Chlorella vulgaris extract | 0.01667 |
| Pueraria lobata root extract | 0.01667 |
| Althaea officinalis root extract | 0.01250 |
| Arnica montana extract | 0.01250 |
| Paeonia suffruticosa root extract | 0.01250 |
| Tilia cordata flower extract | 0.01250 |
| tocopherol | 0.01000 |
| creatin | 0.05000 |
| sodium hyaluronate | 0.100 |
| Arachis hypogaea oil | 0.00050 |
| Simmondsia chinensis seed oil | 0.500 |
| squalane | 0.500 |
| ceramide 3 | 0.00025 |
| triethanolamine | 0.800 |
| polyglyceryl-10-stearate | 0.800 |
| potassium hydroxide | 0.380 |
| phenoxyethanol | 0.300 |
| acrylates/C10-30 alkylacrylate | 0.300 |
| crosspolymer | |
| carbomer | 0.300 |
| hydroxypropylcellulose | 0.100 |
| pentasodium pentetate | 0.05000 |
| disodium EDTA | 0.02000 |
| sodium metabisulfite | 0.02000 |
| retinyl palmitate | 0.00050 |
| behenyl alcohol | 0.00025 |
| polyglyceryl-10 pentastearate | 0.00025 |
| sodium stearoyl lactylate | 0.00025 |
| butylene glycol | 0.100 |

an essence comprising,

| | % by weight |
|---|---|
| water | 74.855 |
| butylene glycol | 5.000 |
| pentylene glycol | 4.000 |
| glycerin | 3.000 |
| PEG-11 methyl ether dimethicone | 2.000 |
| ascorbyl glucoside | 2.000 |
| Fagus sylvatica extract | 2.000 |
| Rosmarinus officinalis leaf extract | 1.000 |
| Arctostaphylos uva ursi leaf extract | 0.500 |
| dipotassium glycyrrhizate | 0.100 |
| Glycine soja extract | 0.500 |
| Scutellaria baicalensis root extract | 0.100 |
| algae extract | 0.300 |
| Morus alba root extract | 0.050 |
| Saxifraga sarmentosa extract | 0.050 |
| Uncaria gambir extract | 0.01000 |
| Aloe barbadensis leaf extract | 0.100 |
| Chlorella vulgaris extract | 0.100 |
| Pueraria lobata root extract | 0.100 |
| Althaea officinalis root extract | 0.01250 |
| Arnica montana extract | 0.01250 |
| Paeonia suffruticosa root extract | 0.01250 |
| Tilia cordata flower extract | 0.01250 |
| tocopherol | 0.01000 |
| creatine | 0.050 |
| sodium hyaluronate | 2.000 |
| potassium hydroxide | 0.625 |
| acrylates/C10-30 alkylacrylate crosspolymer | 0.400 |
| phenoxyethanol | 0.300 |
| styrene/PVP copolymer | 0.200 |
| PEG-50 castor oil | 0.060 |
| pentasodium pentetate | 0.050 |
| polysorbate 20 | 0.040 |
| sodium metabisulfite | 0.150 |
| sodium gluconate | 0.300 |

a toner comprising,

| | |
|---|---|
| water | 80.430 |
| butylene glycol | 5.000 |
| glycerin | 4.000 |
| pentylene glycol | 4.000 |
| PEG-10 methyl ether dimethicone | 2.000 |
| ascorbyl glucoside | 2.000 |
| Fagus sylvatica extract | 0.200 |
| Arctostaphylos uva ursi leaf extract | 0.050 |
| dipotassium glycyrrhizate | 0.050 |
| Glycine soja extract | 0.050 |
| Scutellaria baicalensis root extract | 0.010 |
| algae extract | 0.050 |
| Morus alba root extract | 0.050 |
| Rosmarinus officinalis leaf extract | 0.500 |
| Saxifraga sarmentosa extract | 0.050 |
| Uncaria gambir extract | 0.01000 |
| Aloe barbadensis leaf extract | 0.01667 |
| Chlorella vulgaris extract | 0.01667 |
| Pueraria lobata root extract | 0.01667 |
| Althaea officinalis root extract | 0.01250 |
| Arnica montana extract | 0.01250 |
| Paeonia suffruticosa root extract | 0.01250 |
| Tilia cordata flower extract | 0.01250 |
| tocopherol | 0.01000 |
| sodium hyaluronate | 0.050 |
| pentasodium pentetate | 0.100 |
| sodium gluconate | 0.300 |

a cleansing foam comprising,

| | % by weight |
|---|---|
| water | 25.030 |
| PEG-20 | 20.0000 |
| myristic acid | 14.0000 |
| stearic acid | 14.0000 |
| glycerin | 10.0000 |
| potassium myristoyl glutamate | 7.0000 |
| potassium hydroxide | 6.5000 |
| glycol distearate | 1.0000 |
| Fagus sylvatica extract | 0.200 |
| Arctostaphylos uva ursi leaf extract | 0.050 |
| dipotassium glycyrrhizate | 0.100 |
| Glycine soja extract | 0.050 |
| Scutellaria baicalensis root extract | 0.010 |
| algae extract | 0.0100 |
| Morus alba root extract | 0.0100 |
| Rosmarinus officinalis leaf extract | 0.9000 |
| Saxifraga sarmentosa extract | 0.0100 |
| Uncaria gambir extract | 0.0100 |
| Aloe barbadensis leaf extract | 0.0033 |
| Chlorella vulgaris extract | 0.0033 |
| Pueraria lobata root extract | 0.0033 |
| Althaea officinalis root extract | 0.0025 |
| Arnica montana extract | 0.0025 |
| Paeonia suffruticosa root extract | 0.0025 |
| Tilia cordata flower extract | 0.0025 |
| tocopherol | 0.1000 |
| sodium hyaluronate | 0.1000 |
| Olea europaea fruit oil | 0.5000 |
| butylene glycol | 0.1000 |
| phenoxyethanol | 0.3000 |

and a cleansing gel, comprising,

| | |
|---|---|
| water | 78.364 |
| butylene glycol | 12.000 |
| PEG-7 caprylic/capric glyceride | 4.000 |
| lauryl glucoside | 2.250 |
| carbomer | 1.000 |
| Fagus sylvatica extract | 0.200 |
| Arctostaphylos uva ursi leaf extract | 0.050 |
| dipotassium glycyrrhizate | 0.100 |
| Glycine soja extract | 0.050 |
| Scutellaria baicalensis root extract | 0.010 |
| algae extract | 0.010 |
| Morus alba root extract | 0.010 |
| Rosmarinus officinalis leaf extract | 0.900 |
| Saxifraga sarmentosa extract | 0.010 |
| Uncaria gambir extract | 0.010 |
| Aloe barbadensis leaf extract | 0.00333 |
| Chlorella vulgaris extract | 0.00333 |
| Pueraria lobata root extract | 0.00333 |
| Althaea officinalis root extract | 0.00250 |
| Arnica montana extract | 0.00250 |
| Paeonia suffruticosa root extract | 0.00250 |
| Tilia cordata flower extract | 0.00250 |
| Borago officinalis seed oil | 0.100 |
| Simmondsia chinensis seed oil | 0.100 |
| pentasodium pentetate | 0.050 |
| potassium hydroxide | 0.466 |
| phenoxyethanol | 0.300 |

15. A topical composition, produced by a process comprising:
providing a container;
adding to the container ingredients comprising an Arctostaphylos uva ursi extract, a Scutellaria extract, glycyrrhizic acid, Glycine soja extract, and a Fagus sylvatica extract, wherein the Arctostaphylos uva ursi extract, Scutellaria baicalensis extract, glycyrrhizic acid, Glycine soja extract, and Fagus sylvatica extract are present in amounts effective to inhibit melanogenesis in skin tissue or lighten the color of skin tissue.

16. The topical composition of claim 15, wherein the process of producing the composition further comprises adding to the container a dermatologically acceptable carrier.

17. The topical composition of claim 16, wherein the process of producing the composition further comprises mixing together the ingredients and the dermatologically acceptable carrier.

18. The topical composition of claim 17, wherein the Arctostaphylos uva ursi extract comprises a water extract from a leaf of an Arctostaphylos uva ursi plant, the Scutellaria extract comprises a water extract from a root of a Scutellaria baicalensis plant, the glycyrrhizic acid comprises dipotassium glycyrrhizate, the Glycine soja extract comprises a water extract from a seed of a Glycine soja plant, and the Fagus sylvatica extract comprises a water extract from a bud of a Fagus sylvatica plant.

19. The topical composition of claim 18, wherein the Arctostaphylos uva ursi extract, Scutellaria extract, glycyrrhizic acid, Glycine soja extract, and Fagus sylvatica extract together comprise 0.35% to 3.2% of the topical composition by weight.

20. The topical composition of claim 18, wherein the Arctostaphylos uva ursi extract comprises from 0.05% to 0.5% of the topical composition by weight, the Scutellaria extract comprises from 0.01% to 0.2% of the topical composition by weight, the glycyrrhizic acid comprises from 0.05% to 0.2% of the topical composition by weight, the Glycine soja extract comprises from 0.05% to 0.5% of the topical composition by weight, and Fagus sylvatica extract comprises from 0.2% to 2% of the topical composition by weight.

21. The topical composition of claim 18, wherein the Arctostaphylos uva ursi extract, Scutellaria extract, glycyrrhizic acid, Glycine soja extract, and Fagus sylvatica extract together comprise 1% of the topical composition by weight.

22. The topical composition of claim 21, wherein the Arctostaphylos uva ursi extract, Scutellaria extract, glycyrrhizic acid, Glycine soja extract, and Fagus sylvatica extract are present in equivalent percentages by weight of the topical composition.

23. A method of making a topical composition, comprising:
providing a container;
adding to the container ingredients comprising an Arctostaphylos uva ursi extract, a Scutellaria extract, glycyrrhizic acid, Glycine soja extract, and a Fagus sylvatica extract, wherein the Arctostaphylos uva ursi extract, Scutellaria extract, glycyrrhizic acid, Glycine soja extract, and Fagus sylvatica extract are present in amounts effective to inhibit melanogenesis in skin tissue or lighten the color of skin tissue.

24. The method of claim 23, further comprising adding to the container a dermatologically acceptable carrier.

25. The method of claim 24, further comprising mixing together the ingredients and the dermatologically acceptable carrier.

26. The method of claim 25, wherein the Arctostaphylos uva ursi extract comprises a water extract from a leaf of an Arctostaphylos uva ursi plant, the Scutellaria extract comprises a water extract from a root of a Scutellaria baicalensis plant, the glycyrrhizic acid comprises dipotassium glycyrrhizate, the Glycine soja extract comprises a water extract from a seed of a Glycine soja plant, and the Fagus sylvatica extract comprises a water extract from a bud of a Fagus sylvatica plant.

27. The method of claim 26, wherein the Arctostaphylos uva ursi extract, Scutellaria extract, glycyrrhizic acid, Glycine soja extract, and Fagus sylvatica extract together comprise 0.35% to 3.2% of the topical composition by weight.

28. The method of claim 26, wherein the Arctostaphylos uva ursi extract comprises from 0.05% to 0.5% of the topical composition by weight, the Scutellaria extract comprises from 0.01% to 0.2% of the topical composition by weight, the glycyrrhizic acid comprises from 0.05% to 0.2% of the topical composition by weight, the Glycine soja extract comprises from 0.05% to 0.5% of the topical composition by weight, and Fagus sylvatica extract comprises from 0.2% to 2% of the topical composition by weight.

29. The method of claim 26, wherein the Arctostaphylos uva ursi extract, Scutellaria extract, glycyrrhizic acid, Glycine soja extract, and Fagus sylvatica extract together comprise 1% of the topical composition by weight.

30. The method of claim 29, wherein the Arctostaphylos uva ursi extract, Scutellaria extract, glycyrrhizic acid, Glycine soja extract, and Fagus sylvatica extract comprise equivalent percentages of the topical composition by weight.

31. A method of lightening the color of a subject's skin, comprising applying one or more doses of the topical composition of claim 1 to an area of skin of a subject that has a color, wherein the one or more doses are effective to lighten the color of the area of skin.

32. The method of claim 31, wherein multiple doses of the topical composition are applied to the area of the subject's skin, wherein a first dose is applied to the subject's skin and one or more subsequent doses are applied at a later time or times.

33. The method of claim 31, wherein the application of the topical composition does not decrease the viability of the subject's skin cells in the area of the subject's skin.

34. The method of claim 32, wherein the one or more subsequent doses of the topical composition are applied to the area of the subject's skin until the color of the area of the subject's skin matches the color of a reference area of the subject's skin.

35. The method of claim 32, wherein the area of the subject's skin has an ITA° and the one or more subsequent doses of the topical composition are applied to the area of the subject's skin until the ITA° of the area of the subject's skin has increased by 20% or more.

36. The method of claim 32, wherein an ITA° of the area of the subject's skin is measured and the one or more subsequent doses of the topical composition are applied to the area of the subject's skin until the ITA° of the area of the subject's skin has increased by 20% or more.
